(19) United States
(12) Patent Application Publication
(10) Pub. No.: US 2025/0243221 A1
(43) Pub. Date: Jul. 31, 2025

(54) COVALENT MODIFIERS OF EIF4E INHIBITING COMPOUNDS

(71) Applicant: EFFECTOR THERAPEUTICS, INC., Solana Beach, CA (US)

(72) Inventors: Siegfried H. REICH, La Jolla, CA (US); Paul A. SPRENGELER, Escondido, CA (US)

(73) Assignee: EFFECTOR THERAPEUTICS, INC., Solana Beach, CA (US)

(21) Appl. No.: 17/928,234

(22) PCT Filed: May 24, 2021
(86) PCT No.: PCT/IB2021/054481

§ 371 Date: Aug. 3, 2023

Related U.S. Application Data
(60) Provisional application No. 63/030,408, filed on May 27, 2020.

(51) Int. Cl. C07D 519/00; A61K 31/519

(52) U.S. Cl. CPC C07D 519/00; A61K 31/519

(57) ABSTRACT

The present invention is directed to novel translational inhibitors that bind covalently with eukaryotic initiation factor 4E (eIF4E) and inhibit or modulate the activity of eIF4E, as well as stereoisomers, tautomers and pharmaceutically acceptable salts of such compounds. The present invention also is directed to pharmaceutically acceptable compositions containing such translational inhibitors and associated methods for treating conditions that would benefit from eIF4E inhibition including, but not limited to, treatment of inflammation and various cancers.

### BACKGROUND

Cap-dependent translation initiation in eukaryotes is a highly regulated rate-limiting step, which involves recruitment and assembly of eukaryotic initiation factor 4F (eIF4F), a multiprotein complex on the 5′ cap of the messenger RNA (mRNA). eIF4F consists of at least three proteins: the cap-binding protein eukaryotic initiation factor 4E (eIF4E), the ATP-dependent RNA helicase eukaryotic initiation factor 4A (eIF4A), and the scaffold protein eukaryotic initiation factor 4G (eIF4G). eIF4E directly recognizes the cap structure of mRNAs, and is essential for cap-dependent translation initiation, while eIF4G interacts with the other eIF4F subunits as well as with the poly-A binding protein on the poly-A tail of the mRNA to create a close mRNA circle during translation initiation.

eIF4E is a general translation factor, but it has the potential to enhance preferentially the translation of mRNAs that lead to production of malignancy-associated proteins. This selectivity may relate to an increased requirement for eIF4E and its binding partners for the translation of mRNAs containing extensive secondary structure in their 5′-untranslated regions (5′-UTRs). These mRNAs include those encoding certain proteins that control cell cycle progression and tumorigenesis. Under normal cellular conditions the translation of these malignancy-associated mRNAs is suppressed as the availability of active eIF4E is limited; however, their levels can increase when eIF4E is over-expressed or hyperactivated. Elevated levels of eIF4E have been found in many types of tumors and cancer cell lines including, but not limited to, cancers of the colon, breast, bladder, lung, prostate, gastrointestinal tract, head and neck, Hodgkin's lymphomas and neuroblastomas.

Accordingly, eIF4E has emerged as a potential target for treating several disorders, including cancers. However, while advances have been made in this field there remains a significant need in the art for compounds that specifically inhibit eIF4E activity, particularly with regard to eIF4E's role in regulation of cancer pathways, as well as for associated composition and methods. The present invention fulfills this need and provides further related advantages.

### SUMMARY

The present invention generally relates to compounds having activity as inhibitors that bind covalently to eukaryotic initiation factor 4E (eIF4E), as well as to related compositions and methods for utilizing the inventive compounds as therapeutic agents for treatment of eIF4E-dependent diseases, including the treatment of cancer.

In certain embodiments, the present invention provides a translational inhibitor that binds covalently with eukaryotic initiation factor 4E (eIF4E), or a stereoisomer, tautomer or pharmaceutically acceptable salt of said translational inhibitor.

In some embodiments the present invention provides a pharmaceutical composition comprising (i) a therapeutically effective amount of the translational inhibitor of the invention, or a stereoisomer, a tautomer or a pharmaceutically acceptable salt thereof; and (ii) one or more pharmaceutically acceptable carriers, diluents or excipients.

In other embodiments the present invention provides a translational inhibitor that binds covalently with eukaryotic initiation factor 4E (eIF4E), or a stereoisomer, tautomer or pharmaceutically acceptable salt of said translational inhibitor, wherein the inhibitor has a structure:

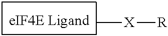

wherein X is a linker selected from a direct bond, alkylene, —O—, —S—, —NH—, —NH(alkylene)-, —NHSO₂—, —NHSO₂(alkylene)-, —C(O)NH—, —C(O)NH(alkylene)-, —C(O)NHSO₂— and —C(O)NHSO₂(alkylene)-, wherein alkyl and alkylene are unsubstituted or substituted; and
R is an electrophilic moiety.

In one embodiment R is alkenyl, aryl, —C(O)₂alkyl, —C(O)₂aryl, —NH(alkenyl), —N(alkyl)(alkenyl), —NHC(O)(alkenyl) or —N(alkyl)C(O)(alkenyl), wherein alkyl, alkenyl and aryl are unsubstituted or substituted.

In yet other embodiments the present invention provides a method for treating a eIF4E-dependent condition in a subject in need thereof comprising administering to the subject (i) a therapeutically effective amount of the translational inhibitor of the invention, or a stereoisomer, tautomer or pharmaceutically acceptable salt thereof, or (ii) a pharmaceutical composition of the invention.

In some other aspects, the present invention provides a method for attenuating or inhibiting the activity of eIF4E in at least one cell overexpressing eIF4E, comprising contacting the at least one cell with the translational inhibitor of the invention, or a stereoisomer, tautomer or pharmaceutically acceptable salt thereof.

In still other aspects, the present invention provides a method for inhibiting translation in at least one cell overexpressing eIF4E, comprising contacting the at least one cell with the translational inhibitor of the invention, or a stereoisomer, tautomer or pharmaceutically acceptable salt thereof.

The above embodiments and other aspects of this invention are readily apparent in the detailed description that follows. Various references are set forth herein which describe in more detail certain background information, procedures and/or compositions, and are each hereby incorporated by reference in their entirety.

### DETAILED DESCRIPTION

In the following description certain specific details are set forth in order to provide a thorough understanding of various embodiments of the invention. However, one skilled in the art will understand that the invention may be practiced without these details. Unless the context requires otherwise, throughout the present specification and claims, the word “comprise” and variations thereof, such as, “comprises” and “comprising” are to be construed in an open, inclusive sense (i.e., as “including, but not limited to”).

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously, which terms and variants thereof are intended to be construed as non-limiting.

In addition, it should be understood that the individual compounds or translational inhibitors, or groups of compounds or translational inhibitors, derived from the various combinations of the structures and substituents described herein, are disclosed by the present application to the same extent as if each compound or translational enhancer, or group of compounds or translational inhibitors was set forth individually. Thus, selection of particular structures or particular substituents is within the scope of the present invention.

The term “consisting essentially of” limits the scope of a claim to the specified materials or steps, or to those that do not materially affect the basic characteristics of a claimed invention. For example, a protein domain, region, or module (e.g., a binding domain, hinge region, linker module) or a protein (which may have one or more domains, regions, or modules) “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

### Definitions

Prior to setting forth this invention in more detail, it may be helpful to an understanding thereof to provide definitions of certain terms to be used herein. Unless defined otherwise, all terms used herein have the same meaning as are commonly understood by one of skill in the art to which this invention belongs. All patents, patent applications and publications referred to throughout the invention herein are incorporated by reference in their entirety. In the event that there is a plurality of definitions for a term herein, those in this section prevail.

As used herein, and unless noted to the contrary, the following terms and phrases have the meaning noted below.

“Amino” refers to the —NH₂ substituent.

“Aminocarbonyl” refers to the —C(O)NH₂ substituent.

“Carboxyl” refers to the —CO₂H substituent.

“Carbonyl” refers to a —C(O)—, —(CO)— or —C(═O)— group. All notations are used interchangeably within the specification.

“Cyano” refers to the —C≡N substituent.

“Acetyl” refers to the —C(O)CH₃ substituent.

“Hydroxy” or “hydroxyl” refers to the —OH substituent.

“Hydroxyalkylene” refers to the -(alkylene)OH substituent.

“Oxo” refers to a ═O substituent.

“Thio” or “thiol” refer to a —SH substituent.

“Alkyl” refers to a saturated, straight or branched hydrocarbon chain radical consisting solely of carbon and hydrogen atoms, having from one to twelve carbon atoms (C₁-C₁₂ alkyl), from one to eight carbon atoms (C₁-C₈ alkyl) or from one to six carbon atoms (C₁-C₆ alkyl), and which is attached to the rest of the molecule by a single bond. Exemplary alkyl groups include methyl, ethyl, n-propyl, 1-methylethyl (iso-propyl), n-butyl, n-pentyl, 1,1-dimethylethyl (t-butyl), 3-methylhexyl, 2-methylhexyl, and the like.

“Lower alkyl” has the same meaning as alkyl defined above but having from one to four carbon atoms (C₁-C₄ alkyl).

“Alkenyl” refers to an unsaturated alkyl group having at least one double bond and from two to twelve carbon atoms (C₂-C₁₂ alkenyl), from two to eight carbon atoms (C₂-C₅ alkenyl) or from two to six carbon atoms (C₂-C₆ alkenyl), and which is attached to the rest of the molecule by a single bond, e.g., ethenyl, propenyl, butenyl, pentenyl, hexenyl, and the like.

“Alkynyl” refers to an unsaturated alkyl group having at least one triple bond and from two to twelve carbon atoms (C₂-C₁₂ alkynyl), from two to ten carbon atoms (C₂-C₁₀ alkynyl) from two to eight carbon atoms (C₂-C₈ alkynyl) or from two to six carbon atoms (C₂-C₆ alkynyl), and which is attached to the rest of the molecule by a single bond, e.g., ethynyl, propynyl, butynyl, pentynyl, hexynyl, and the like.

“Alkylene” or “alkylene chain” refers to a straight or branched divalent hydrocarbon (alkyl) chain linking the rest of the molecule to a radical group, consisting solely of carbon and hydrogen, respectively. Alkylenes can have from one to twelve carbon atoms, e.g., methylene, ethylene, propylene, n-butylene, and the like. The alkylene chain is attached to the rest of the molecule through a single or double bond. The points of attachment of the alkylene chain to the rest of the molecule can be through one carbon or any two carbons within the chain. “Optionally substituted alkylene” refers to alkylene or substituted alkylene.

“Alkenylene” refers to divalent alkene. Examples of alkenylene include without limitation, ethenylene (—CH═CH—) and all stereoisomeric and conformational isomeric forms thereof. “Substituted alkenylene” refers to divalent substituted alkene. “Optionally substituted alkenylene” refers to alkenylene or substituted alkenylene.

“Alkynylene” refers to divalent alkyne. Examples of alkynylene include without limitation, ethynylene, propynylene. “Substituted alkynylene” refers to divalent substituted alkyne.

“Alkoxy” refers to a radical of the formula —ORₐ where Rₐ is an alkyl having the indicated number of carbon atoms as defined above. Examples of alkoxy groups include without limitation —O-methyl (methoxy), —O-ethyl (ethoxy), —O-propyl (propoxy), —O-isopropyl (iso propoxy) and the like.

“Aryl” refers to a hydrocarbon ring system radical comprising hydrogen, 6 to 18 carbon atoms and at least one aromatic ring. Exemplary aryls are hydrocarbon ring system radical comprising hydrogen and 6 to 9 carbon atoms and at least one aromatic ring; hydrocarbon ring system radical comprising hydrogen and 9 to 12 carbon atoms and at least one aromatic ring; hydrocarbon ring system radical comprising hydrogen and 12 to 15 carbon atoms and at least one aromatic ring; or hydrocarbon ring system radical comprising hydrogen and 15 to 18 carbon atoms and at least one aromatic ring. For purposes of the compounds of the present invention, the aryl radical may be a monocyclic, bicyclic, tricyclic or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. “Optionally substituted aryl” refers to an aryl group or a substituted aryl group.

“Cycloalkyl” refers to a stable non-aromatic monocyclic or polycyclic hydrocarbon radical consisting solely of carbon and hydrogen atoms, which may include fused or bridged ring systems, having from three to fifteen carbon atoms, preferably having from three to ten carbon atoms, three to nine carbon atoms, three to eight carbon atoms, three to seven carbon atoms, three to six carbon atoms, three to five carbon atoms, a ring with four carbon atoms, or a ring with three carbon atoms. The cycloalkyl ring may be saturated or unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7-dimethyl-bicyclo[2.2.1]heptanyl, and the like.

“Fused” refers to any ring structure described herein which is fused to an existing ring structure in the compounds or translational enhancers of the present invention. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” or “halogen” refers to bromo (bromine), chloro (chlorine), fluoro (fluorine), or iodo (iodine).

“Haloalkyl” refers to an alkyl radical having the indicated number of carbon atoms, as defined herein, wherein one or more hydrogen atoms of the alkyl group are substituted with a halogen (halo radicals), as defined above. The halogen atoms can be the same or different. Exemplary haloalkyls are trifluoromethyl, difluoromethyl, trichloromethyl, 2,2,2-trifluoroethyl, 1,2-difluoroethyl, 3-bromo-2-fluoropropyl, 1,2-dibromoethyl, and the like.

“Heterocyclyl,” “heterocycle,” or “heterocyclic ring” refers to a stable 3- to 18-membered saturated or unsaturated radical which consists of two to twelve carbon atoms and from one to six heteroatoms, for example, one to five heteroatoms, one to four heteroatoms, one to three heteroatoms, or one to two heteroatoms selected from the group consisting of nitrogen, oxygen and sulfur. Exemplary heterocycles include without limitation stable 3-15 membered saturated or unsaturated radicals, stable 3-12 membered saturated or unsaturated radicals, stable 3-9 membered saturated or unsaturated radicals, stable 8-membered saturated or unsaturated radicals, stable 7-membered saturated or unsaturated radicals, stable 6-membered saturated or unsaturated radicals, or stable 5-membered saturated or unsaturated radicals.

Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of non-aromatic heterocyclyl radicals include, but are not limited to, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, thietanyl, trithianyl, tetrahydropyranyl, thiomorpholinyl, thiamorpholinyl, 1-oxo-thiomorpholinyl, and 1,1-dioxo-thiomorpholinyl. Heterocyclyls include heteroaryls as defined herein, and examples of aromatic heterocyclyls are listed in the definition of heteroaryls below.

“Heteroaryl” or “heteroarylene” refers to a 5- to 14-membered ring system radical comprising hydrogen atoms, one to thirteen carbon atoms, one to six heteroatoms selected from the group consisting of nitrogen, oxygen and sulfur, and at least one aromatic ring. For purposes of the compounds or translational enhancers of the present invention, the heteroaryl radical may be a stable 5-12 membered ring, a stable 5-10 membered ring, a stable 5-9 membered ring, a stable 5-8 membered ring, a stable 5-7 membered ring, or a stable 6 membered ring that comprises at least 1 heteroatom, at least 2 heteroatoms, at least 3 heteroatoms, at least 4 heteroatoms, at least 5 heteroatoms or at least 6 heteroatoms. Heteroaryls may be a monocyclic, bicyclic, tricyclic or tetracyclic ring system, which may include fused or bridged ring systems; and the nitrogen, 2 carbon or sulfur atoms in the heteroaryl radical may be optionally oxidized; the nitrogen atom may be optionally quaternized. The heteroatom may be a member of an aromatic or non-aromatic ring, provided at least one ring in the heteroaryl is aromatic. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl).

“Thioalkyl” refers to a radical of the formula —SRₐ where Rₐ is an alkyl radical as defined above containing one to twelve carbon atoms, at least 1-10 carbon atoms, at least 1-8 carbon atoms, at least 1-6 carbon atoms, or at least 1-4 carbon atoms.

“Thione” refers to a ═S group attached to a carbon atom of a saturated or unsaturated (C₃-C₈)cyclic or a (C₁-C₈)acyclic moiety.

“Sulfoxide” refers to a —S(O)— group in which the sulfur atom is covalently attached to two carbon atoms.

“Sulfone” refers to a —S(O)₂— group in which a hexavalent sulfur is attached to each of the two oxygen atoms through double bonds and is further attached to two carbon atoms through single covalent bonds.

The compounds or translational enhancers provided in the present invention can exist in various isomeric forms, as well as in one or more tautomeric forms, including both single tautomers and mixtures of tautomers. The term “isomer” is intended to encompass all isomeric forms of a compound of the present invention, including tautomeric forms of the compound.

Some compounds or translational enhancers described herein can have asymmetric centers and therefore exist in different enantiomeric and diastereomeric forms. A compound provided in the present invention can be in the form of an optical isomer or a diastereomer. Accordingly, the invention encompasses compounds or translational enhancers provided in the present invention and their uses as described herein in the form of their optical isomers, diastereoisomers and mixtures thereof, including a racemic mixture. Optical isomers of the compounds or translational enhancers provided in the present invention can be obtained by known techniques such as asymmetric synthesis, chiral chromatography, or via chemical separation of stereoisomers through the employment of optically active resolving agents.

Unless otherwise indicated, “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds or translational enhancers are prepared as single enantiomers from the methods used to prepare them.

In this description, a “pharmaceutically acceptable salt” is a pharmaceutically acceptable, organic or inorganic acid or base salt of a compound of the present invention. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

In addition, it should be understood that the individual compounds or translational inhibitors, or groups of compounds or translational inhibitors, derived from the various combinations of the structures and substituents described herein, are disclosed by the present application to the same extent as if each compound or translational inhibitor or group of compounds or translational inhibitors was set forth individually. Thus, selection of particular structures or particular substituents is within the scope of the present invention.

As used herein, the term “derivative” refers to a modification of a compound by chemical or biological means, with or without an enzyme, which modified compound is structurally similar to a parent compound and (actually or theoretically) derivable from that parent compound. Generally, a “derivative” differs from an “analog” in that a parent compound may be the starting material to generate a “derivative,” whereas the parent compound may not necessarily be used as the starting material to generate an “analog.” A derivative may have different chemical, biological or physical properties from the parent compound, such as being more hydrophilic or having altered reactivity as compared to the parent compound. Derivatization (i.e., modification) may involve substitution of one or more moieties within the molecule (e.g., a change in functional group). For example, a hydrogen may be substituted with a halogen, such as fluorine or chlorine, or a hydroxyl group (—OH) may be replaced with a carboxylic acid moiety (—COOH). Other exemplary derivatizations include glycosylation, alkylation, acylation, acetylation, ubiqutination, esterification, and amidation.

The term “derivative” also refers to all solvates, for example, hydrates or adducts (e.g., adducts with alcohols), active metabolites, and salts of a parent compound. The type of salt depends on the nature of the moieties within the compound. For example, acidic groups, such as carboxylic acid groups, can form alkali metal salts or alkaline earth metal salts (e.g., sodium salts, potassium salts, magnesium salts, calcium salts, and also salts with physiologically tolerable quaternary ammonium ions and acid addition salts with ammonia and physiologically tolerable organic amines such as, for example, triethylamine, ethanolamine or tris-(2-hydroxyethyl)amine). Basic groups can form acid addition salts with, for example, inorganic acids such as hydrochloric acid, sulfuric acid or phosphoric acid, or with organic carboxylic acids or sulfonic acids such as acetic acid, citric acid, lactic acid, benzoic acid, maleic acid, fumaric acid, tartaric acid, methanesulfonic acid or p-toluenesulfonic acid. Molecules that simultaneously contain a basic group and an acidic group, for example, a carboxyl group in addition to basic nitrogen atoms, can be present as zwitterions. Salts can be obtained by customary methods known to those skilled in the art, for example, by combining a compound with an inorganic or organic acid or base in a solvent or diluent, or from other salts by cation exchange or anion exchange.

As used herein, the term “eIF4E,” also referred to as “eukaryotic translation initiation factor 4E,” refers to a translation initiation factor that, when part of an eIF4F pre-initiation complex also comprising eIF4A RNA helicase and eIF4G scaffold protein, binds to the 7-methyl-guanosine (m7GpppX) 5′-cap structure on eukaryotic mRNAs and directs ribosomes to the cap structure. The availability of eIF4E as part of the eIF4F complex is a limiting factor in controlling the rate of translation. Interactions of eIF4E and the m⁷G cap and eIF4G are tightly regulated by key mitogenic signals, such as the PI3K/mTOR and Ras/MAPK signal transduction pathways. There are four different isoforms of eIF4E: isoform 1 is the canonical sequence; isoform 2 contains an alternate in-frame exon in the 3′-coding region compared to isoform 1; isoform 3 uses an alternate 5′-terminal exon, which results in a different 5′-UTR and use of an alternate translation start codon compared to isoform 1; and isoform 4 differs in its 5′-UTR and contains an alternate exon in its 5′-coding region compared to isoform 1. In certain embodiments, eIF4E refers to eIF4E isoform 1, isoform 2, isoform 3, isoform 4, or any combination thereof. In certain embodiments, eIF4E refers to the canonical eIF4E isoform 1. In particular embodiments, eIF4E refers to human eIF4E.

As used herein, the terms “bind,” “attached,” “linked,” “connected,” “bound”, “coupled,” “fused,” or “fusion” are used interchangeably. These terms refer to the joining together of two more elements or components, by whatever means including chemical conjugation and recombinant means.

As used herein, the term “attached directly” refers to two molecules that are attached or linked or connected or bound or coupled or fused to one another in the absence of an intervening molecule.

As used herein, the terms “translation” or “translation reaction” refer to the process or mechanism of synthesizing a protein from an mRNA.

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to chemical entities that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical entities that have a structure that is different from the general chemical structure of an amino acid, but that functions in a manner similar to a naturally occurring amino acid.

Each of the 20 most common amino acids has its specific chemical characteristics and its unique role in a protein structure and function. For example, based on the propensity of the side chain to be in contact with water, amino acids are classified as “hydrophobic” (low propensity to be in contact with water), “polar” and “charged” (energetically favorable contact with water). The “charged amino acids” include two basic amino acids, lysine and arginine (+charge), and two acidic amino acids, aspartic acid and glutamic acid (− charge). “Polar amino acids” include serine and threonine (contain a hydroxyl group), asparagine and glutamine (contain amide group), tyrosine, and cysteine. Histidine is also a polar residue, although its behavior depends on the polarity of its environment.

As used herein, “protein” or “polypeptide” refers to a polymer of amino acid residues. Proteins apply to naturally occurring amino acid polymers, as well as to amino acid polymers in which one or more amino acid residue is an artificial chemical mimetic of a corresponding naturally occurring amino acid and non-naturally occurring amino acid polymers.

“Nucleic acid molecule” or “polynucleotide” refers to a polymeric compound including covalently linked nucleotides, which can be made up of natural subunits (e.g., purine or pyrimidine bases) or non-natural subunits (e.g., morpholine ring). Purine bases include adenine, guanine, hypoxanthine, and xanthine, and pyrimidine bases include uracil, thymine, and cytosine. Nucleic acid molecules include polyribonucleic acid (RNA), polydeoxyribonucleic acid (DNA), which includes cDNA, genomic DNA, and synthetic DNA, either of which may be single or double stranded. If single stranded, the nucleic acid molecule may be the coding strand or non-coding (anti-sense strand). A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing.

The term “inhibit” or “inhibitor” refers to an alteration, interference, reduction, down regulation, blocking, suppression, abrogation or degradation, directly or indirectly, in the expression, amount or activity of a target gene, target protein, or signaling pathway relative to (1) a control, endogenous or reference target or pathway, or (2) the absence of a target or pathway, wherein the alteration, interference, reduction, down regulation, blocking, suppression, abrogation or degradation is statistically, biologically, or clinically significant.

As used herein, the terms “inhibit,” “inhibitor” and the like refer to the ability of a translational inhibitor of the invention to decrease the function, or activity of, for example, eukaryotic initiation factor 4E (eIF4E). “Inhibition”, in its various forms, is intended to encompass inhibition, antagonism, or partial antagonism of the biological activity or function of eIF4E. Translational inhibitors are compounds that bind to, partially or totally block stimulation, decrease, prevent, delay activation, inactivate, desensitize, or down regulate signal transduction. The ability of a translational inhibitor to inhibit the function or biological activity of eIF4E can be demonstrated in an enzymatic assay or a cell-based assay.

The term “irreversibly binds,” as used herein, refers to the fact that a translational inhibitor of the invention, upon contact with a target protein (e.g., eIF4E) causes the formation of a new covalent bond with or within the protein, whereby one or more of the target protein's biological activities or function is diminished or abolished notwithstanding the subsequent presence or absence of the translational inhibitor.

The term “selectively binds” or “specifically bind,” as used herein, refers to the ability of a translational inhibitor of the invention to bind to a target protein, such as, for example, eIF4E with greater affinity than it binds to a non-target protein. In certain embodiments, selective or specific binding refers to binding of a translational inhibitor of the invention to a target protein with an affinity that is at least 10, 50, 100, 250, 500, 1000 or more times greater than the affinity for a non-target protein.

“Treatment,” “treating” or “ameliorating” refers to medical management of a disease, disorder, or condition of a subject (i.e., patient), which may be therapeutic, prophylactic/preventative, or a combination treatment thereof. A treatment may improve or decrease the severity at least one symptom of a disease, delay worsening or progression of a disease, or delay or prevent onset of additional associated diseases. “Reducing the risk of developing a disease” refers to preventing or delaying onset of a disease or reoccurrence of one or more symptoms of the disease (e.g., cancer). In certain embodiments, the immune modulation provided by the translational enhancers of this invention aids or augments treatment regimens or aids or augments a host organism's immune system.

A “patient” or “subject” includes an animal, such as a human, cow, horse, sheep, lamb, pig, chicken, turkey, quail, cat, dog, mouse, rat, rabbit or guinea pig. The animal can be a mammal, such as a non-primate and a primate (e.g., monkey and human). In some embodiments, a subject is a human, such as a human infant, child, adolescent or adult.

Further, a “mammal” includes primates, such as humans, monkeys and apes, and non-primates such as domestic animals, including laboratory animals and household pets (e.g., cats, dogs, swine, cattle, sheep, goats, horses, rabbits), and non-domestic animals, such as wildlife or the like.

“Effective amount” or “therapeutically effective amount” refers to that amount of a composition described herein which, when administered to a mammal (e.g., human), is sufficient to aid in treating a disease. The amount of a composition that constitutes a “therapeutically effective amount” will vary depending on the cell preparations, the condition and its severity, the manner of administration, and the age of the mammal to be treated, but can be determined routinely by one of ordinary skill in the art having regard to his own knowledge and to this invention. When referring to an individual active ingredient or composition, administered alone, a therapeutically effective dose refers to that ingredient or composition alone. When referring to a combination, a therapeutically effective dose refers to combined amounts of the active ingredients, compositions or both that result in the therapeutic effect, whether administered serially, concurrently or simultaneously.

The term “therapeutically effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

The terms “co-administration” and the like, as used herein, are meant to encompass administration of two or more selected therapeutic agents to a single subject, and are intended to include treatment regimens in which the agents are administered by the same or different route of administration or at the same or different time.

As used herein, “hyperproliferative disorder” or “hyperproliferative disease” refers to excessive growth or proliferation as compared to a normal cell or an undiseased cell. Exemplary hyperproliferative disorders include dysplasia, neoplasia, non-contact inhibited or oncogenically transformed cells, tumors, cancers, carcinoma, sarcoma, malignant cells, pre-malignant cells, as well as non-neoplastic or non-malignant hyperproliferative disorders (e.g., adenoma, fibroma, lipoma, leiomyoma, hemangioma, fibrosis, restenosis, or the like). In certain embodiments, a cancer being treated by the compositions and methods of this invention includes carcinoma (epithelial), sarcoma (connective tissue), lymphoma or leukemia (hematopoietic cells), germ cell tumor (pluripotent cells), blastoma (immature “precursor” cells or embryonic tissue), or any combination thereof. These various forms of hyperproliferative disease are known in the art and have established criteria for diagnosis and classification (e.g., Hanahan and Weinberg, Cell 144:646, 2011; Hanahan and Weinberg Cell 100:57, 2000; Cavallo et al., Canc. Immunol. Immunother. 60:319, 2011; Kyrigideis et al., J. Carcinog. 9:3, 2010).

“Isomerism” means compounds or translational enhancers that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

A carbon atom bonded to four nonidentical substituents is termed a “chiral center.” “Chiral isomer” means a compound with at least one chiral center. Compounds or translational enhancers with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

The term “tautomer” refers to a proton shift from one atom of a molecule to another atom of the same molecule. A tautomer is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertable by tautomerizations is called tautomerism.

It is to be understood that the compounds or translational inhibitors of the present invention may be depicted as different tautomers. It should also be understood that when compounds or translational inhibitors have tautomeric forms, all tautomeric forms are intended to be included in the scope of the present invention, and the naming of the compounds or translational inhibitors does not exclude any tautomer form. It will be understood that certain tautomers may have a higher level of activity than others.

In the present specification, the structural formula of a translational inhibitor of the invention represents a certain isomer for convenience in some cases, but the present invention includes all isomers, such as geometrical isomers, optical isomers based on an asymmetrical carbon, stereoisomers, tautomers, and the like, it being understood that not all isomers may have the same level of activity. In addition, in certain cases, a crystal polymorphism may be present for the translational inhibitors of the invention. It is noted that any crystal form, crystal form mixture, or anhydride or hydrate thereof is included in the scope of the present invention.

The terms “comprising”, “having”, “being of” as in “being of a chemical formula”, “including”, and “containing” are to be construed as open terms (i.e., meaning “including but not limited to”) unless otherwise noted. Additionally whenever “comprising” or another open-ended term is used in various embodiments, it is to be understood that the same embodiment can be more narrowly claimed using the intermediate term “consisting essentially of or the closed term “consisting of.”

As used herein, the expressions “one or more of A, B, or C,” “one or more A, B, or C,” “one or more of A, B, and C,” “one or more A, B, and C” and the like are used interchangeably and all refer to a selection from the group consisting of A, B, and/or C, i.e., one or more As, one or more Bs, one or more Cs, or any combination thereof.

As used herein, the term “translational inhibitor” refers to any compound or biological molecule that decreases the rate, or amount, or both of polypeptide or protein production from an mRNA. A translational inhibitor of the invention provides decreased or attenuated polypeptide or protein production in vitro or in the cell from an mRNA comprising the translational inhibitor, preferably less efficiently than a cell not comprising the translational enhancer of the invention. A translational inhibitor may decrease or attenuate polypeptide or protein production from mRNAs, e.g., by decreasing stability/half-life/bioavailability/biodistribution of endogenous and/or exogenous mRNAs, decreasing the translation efficiency of endogenous and/or exogenous mRNAs, increasing an immune response to exogenous mRNAs, and/or decreasing delivery and/or permeability of exogenous mRNA molecules to cells.

As used herein, the phrases “translational inhibitor of the invention” and “translational inhibitors of the invention” are used interchangeably to refer to a compound that binds covalently with eukaryotic initiation factor 4E (eIF4E) and inhibits the activity or function of eIF4E, or a stereoisomer, tautomer or pharmaceutically acceptable salt of said translational inhibitor. In certain aspects, covalent binding of the translational inhibitor with eIF4E is reversible. In other aspects, covalent binding of the translational inhibitor with eIF4E is irreversible.

The terms “eIF4E ligand,” “ligand,” and “compound” are used interchangeably to refer to any compound or biological molecule that binds to the eukaryotic translation initiation factor 4E (eIF4E). In certain embodiments, binding of the eIF4E ligand to eIF4E affects the interaction of eIF4E with any of the other components of the cellular translational machinery (e.g., other translation initiation factors). In certain aspects, binding of the eIF4E ligand to eIF4E decreases or eliminates the interaction of eIF4E with one or more components of the cellular translational machinery.

As used herein, the term “linker,” or “linker molecule,” or “linking group” means an organic moiety that connects two parts of a compound.

As used herein, the term “moiety” refers to a specific segment or functional group of a molecule. Chemical moieties are often recognized chemical entities embedded in or appended to a molecule. In certain aspects, a translational inhibitor of the invention comprises an eIF4E ligand attached by a linker to a chemical moiety that binds covalently with eIF4E. Thus, in certain embodiments, a translational inhibitor of the invention covalently binds with an amino acid residue near the active site of eIF4E via the chemical moiety. In other embodiments, a translational inhibitor of the invention covalently binds with an amino acid residue at the active site of eIF4E via the chemical moiety. In specific embodiments, a translational inhibitor of the invention covalently binds with an amino acid residue at or near the active site of eIF4E via the chemical moiety. In certain aspects, covalent binding of the translational inhibitor with eIF4E is revesible. In other aspects, covalent binding of the translational inhibitor with eIF4E is irrevesible.

As used herein, “treating” or “treat” describes the management and care of a patient for the purpose of combating a disease, condition, or disorder and includes the administration of an active ingredient of the invention to alleviate the symptoms or complications of a disease, condition or disorder, or to eliminate the disease, condition or disorder. The term “treat” can also include treatment of a cell in vitro or an animal model.

An active ingredient of the invention, can or may also be used to prevent a relevant disease, condition or disorder, or used to identify suitable candidates for such purposes. As used herein, “preventing,” “prevent,” or “protecting against” or “ameliorating,” describes reducing or eliminating the onset of the symptoms or complications of such disease, condition or disorder.

A “pharmaceutical composition” is a formulation containing the translational inhibitor of the invention in a form suitable for administration to a subject. In some embodiments, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a translational inhibitor of the invention include powders, sprays, ointments, pastes, creams, lotions, gels, solutions, patches and inhalants. In some embodiments, the translational inhibitors of the invention are mixed under sterile conditions with a pharmaceutically acceptable carrier, and with any preservatives, buffers, or propellants that are required.

As used herein, the phrase “pharmaceutically acceptable” refers to those compounds, translational inhibitors, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

An “effective amount” of the translational inhibitors disclosed herein is based, at least in part, on the target tissue, target cell type, means of administration, physical characteristics of the polynucleotide (e.g., size, and extent of modified nucleosides) and other components of the multimeric structures, and other determinants. In general, an effective amount of a translational inhibitor of the invention provides a decreased or attenuated polypeptide or protein production in the cell, preferably less efficiently than a cell not comprising the translational enhancer of the invention. Decreased polypeptide production may be demonstrated by increased cell transfection (i.e., the percentage of cells transfected with the multimeric structures), decreased protein translation from the polynucleotide, increased nucleic acid degradation (as demonstrated, e.g., by increased duration of protein translation from a modified polynucleotide), or decreased polypeptide production in the host cell.

### Translational Inhibitors of the Invention

In certain embodiments, the invention provides a translational inhibitor that binds covalently with eukaryotic initiation factor 4E (eIF4E). In other embodiments, the invention provides a stereoisomer, tautomer or pharmaceutically acceptable salt of the translational inhibitors of the invention. In certain embodiments, the translational inhibitor is an inhibitor of eIF4E. In certain embodiments, the translational inhibitor inhibits the biological activity of eIF4E. In other embodiments, the translational inhibitor inhibits the function of eIF4E (i.e., the translational inhibitor inhibits interaction of eIF4E with one or more components of the cellular translational machinery including, but not limited to, one or more translational initiation factors, poly-A binding protein, ribosomal subunits, and the like).

In some embodiments, the translational inhibitors of the invention bind covalently with eIF4E. In certain embodiments, covalent binding of the translational inhibitors of the invention with eIF4E is reversible. In other embodiments, covalent binding of the translational inhibitors of the invention with eIF4E is irreversible. In certain aspects, the translational inhibitors of the invention bind covalently and reversibly with eIF4E. In other aspects, the translational inhibitors of the invention bind covalently and irreversibly with eIF4E. In additional aspects, the translational inhibitors of the invention selectively bind with eIF4E. In some aspects, the translational inhibitors of the invention selectively reversibly bind with eIF4E. In other aspects, the translational inhibitors of the invention selectively irreversibly bind with eIF4E.

In certain embodiments, a translational inhibitor of the invention covalently binds with an amino acid residue near the active site of eIF4E. In certain embodiments, covalent binding of a translational inhibitor of the invention with an amino acid residue near the active site of eIF4E is reversible. In other embodiments, covalent binding of a translational inhibitor of the invention with an amino acid residue near the active site of eIF4E is irreversible. In certain aspects, a translational inhibitor of the invention covalently and reversibly binds with an amino acid residue near the active site of eIF4E. In additional aspects, a translational inhibitor of the invention covalently and irreversibly binds with an amino acid residue near the active site of eIF4E.

In other embodiments, a translational inhibitor of the invention covalently binds with an amino acid residue at the active site of eIF4E. In certain embodiments, covalent binding of a translational inhibitor of the invention with an amino acid residue at the active site of eIF4E is reversible. In other embodiments, covalent binding of a translational inhibitor of the invention with an amino acid residue at the active site of eIF4E is irreversible. In certain aspects, a translational inhibitor of the invention covalently and reversibly binds with an amino acid residue at the active site of eIF4E. In other aspects, a translational inhibitor of the invention covalently and irreversibly binds with an amino acid residue at the active site of eIF4E.

In certain aspects, the amino acid residue at or near an active site of eIF4E is a charged amino acid residue or a polar amino acid residue. Charged amino acids include, without limitation, lysine, arginine, aspartic acid, and glutamic acid. Polar amino acids include, without limitation, serine, threonine, asparagine, glutamine, tyrosine, cysteine, and histidine.

In certain aspects, the amino acid residue at or near an active site of eIF4E includes, but is not limited to, a cysteine residue, a lysine residue, a serine residue, or an arginine residue. In some aspects, the amino acid residue at or near an active site of eIF4E is a cysteine residue. In other aspects, the amino acid residue at or near an active site of eIF4E is a lysine residue. In yet other aspects, the amino acid residue at or near an active site of eIF4E is an arginine residue. In still other aspects, the amino acid residue at or near an active site of eIF4E is a serine residue.

In one embodiment the present invention provides a translational inhibitor that binds covalently with eukaryotic initiation factor 4E (eIF4E), or a stereoisomer, tautomer or pharmaceutically acceptable salt of said translational inhibitor, wherein the inhibitor has a structure:

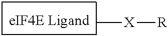

wherein X is a linker selected from a direct bond, alkylene, —O—, —S—, —NH—, —NH(alkylene)-, —NHSO₂—, —NHSO₂(alkylene)-, —C(O)NH—, —C(O)NH(alkylene)-, —C(O)NHSO₂— and —C(O)NHSO₂(alkylene)-, wherein alkyl and alkylene are unsubstituted or substituted; and
R is an electrophilic moiety.

In one embodiment R is alkenyl, aryl, —C(O)₂alkyl, —C(O)₂aryl, —NH(alkenyl), —N(alkyl)(alkenyl), —NHC(O)(alkenyl) or —N(alkyl)C(O)(alkenyl), wherein alkyl, alkenyl and aryl are unsubstituted or substituted.

In another embodiment R is alkenyl, —C(O)₂aryl or —N(alkyl)C(O)(alkenyl), wherein aryl and alkenyl are optionally substituted with halogen.

In one embodiment, the translational inhibitor of the invention comprises an eIF4E Ligand having a structure according to Formula I:

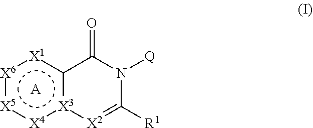

X¹ is CR², —C-L¹-Y or N;
X², X⁵ and X⁶ are independently CR² or N,
   wherein X⁵ and X⁶ together with 3 or 4 carbon or nitrogen atoms combine to form a 5- or 6-membered cycloalkyl or heterocyclyl,
   or when X² is CR², R¹ and R² together with the atoms they attached to form a 6-membered aryl or heteroaryl;
X³ is C, or X³ is C or N when X⁴ is a bond;
X⁴ is a bond, CR² or N,
   wherein X⁴ and X⁵ together with 3 or 4 carbon or nitrogen atoms combine to form a 5- or 6-membered heteroaryl;
Q is H or -L¹-Y;
L¹ is —(CH₂)—, —(CH₂)₂—, —(CH₂)₃—, —CH((C₁-C₈)alkyl)(CH₂)—, —CH((C₁-C₈)alkyl)(CH₂)₂—, —(CH₂)₂—O—, —CH₂CH═CH—, —CH₂C≡C— or —CH₂(cyclopropyl)-;
Y is

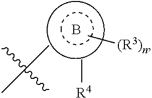

wherein
Ring B is a six-membered aryl, heteroaryl or heterocyclyl;
R¹ is H, OH, halo, CN, (C₁-C₈)alkyl, (C₁-C₈)haloalkyl, (C₃-C₆)cycloalkyl or NR⁵R⁵;
R² is independently H, halo, CN, NO, NO₂, C≡H, (C₁-C₈)alkyl, (C₁-C₈)haloalkyl, CH₂SR⁵, OR⁵, NHR⁵, NR⁵R⁵, [(C₁-C₈)alkylene]heterocyclyl, [(C₁-C₈)alkylene]heteroaryl, [(C₁-C₈)alkylene]NHR⁵, [(C₁-C₈)alkylene]NR⁵R⁵, [(C₁-C₈)alkylyne]NR⁵R⁵, C(O)R⁵, C(O)OR⁵, C(O)NHR⁵, C(O)NR⁵R⁵, SR⁵, S(O)R⁵, SO₂R⁵, SO₂NHR⁵, SO₂NR⁵R⁵, NH(CO)R⁶, NR⁵(CO)R⁶, aryl, heteroaryl, cycloalkyl or heterocyclyl;
R³ is independently OH, halo, CN, NO₂, (C₁-C₆)alkyl, (C₁-C₆)haloalkyl, (C₁-C₆)alkoxy, C≡H, NHR⁷, NR⁷R⁷, CO₂H, CO₂R⁷, [(C₁-C₃)alkylene](C₁-C₃)alkoxy, [(C₁-C₃)alkylene]CO₂H, (C₃-C₅)cycloalkyl, ═O. ═S, SR⁷, SO₂R⁷, NH(CO)R⁷ or NR⁷(CO)R⁷;
R⁴ is H, OH, halo, CN, (C₁-C₃)alkyl, (C₁-C₃)haloalkyl, (C₁-C₃)alkoxy, SR⁷ or Z, wherein Z is

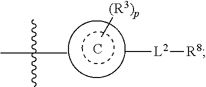

Ring C is cycloalkyl, heterocyclyl, aryl or heteroaryl;
L² is —C(R⁶)(R⁶)—, —C(R⁶)(R⁶)C(R⁶)(R⁶)—, —C(R⁶)═C(R⁶)—, —N(R⁵)C(R⁶)(R⁶)—, —OC(R⁶)(R⁶)—, —C(═O)—, —C(═O)N(R⁵)C(R⁶)(R⁶)— or a bond;
R⁵ is independently H, (C₁-C₃)alkyl, (C₁-C₃)haloalkyl, (C₃-C₅)cycloalkyl, CO₂H, [(C₁-C₃)alkylene]heteroaryl, [(C₁-C₃)alkylene]aryl, [(C₁-C₃)alkylene]CO₂H, heterocyclyl, aryl or heteroaryl,
   or wherein two R⁵ substituents together with a nitrogen atom form a 4-, 5-, 6- or 7-membered heterocyclyl;
R⁶ is independently H, OH, halo, CN, (C₁-C₃)alkyl, (C₁-C₃)haloalkyl, (C₁-C₃)alkoxy, NHR⁷, NR⁷R⁷, CO₂H, [(C₁-C₃)alkylene]CO₂H, (C₃-C₅)cycloalkyl, SR⁷, NH(CO)R⁷ or NR⁷(CO)R⁷;
R⁷ is independently H, (C₁-C₈)alkyl, (C₁-C₈)haloalkyl, cycloalkyl, heterocyclyl, aryl or heteroaryl;
R⁸ is H, OH, CO₂H, CO₂R⁷, CF₂C(R⁶)₂OH, C(R⁶)₂OH, C(CF₃)₂OH, SO₂H, SO₃H, CF₂SO₂C(R⁶)₃, CF₂SO₂N(H)R⁵, SO₂N(H)R⁵, SO₂N(H)C(O)R⁶, C(O)N(H)SO₂R⁵, C(O)haloalkyl, C(O)N(H)OR⁵, C(O)N(R⁵)OH, C(O)N(H)R⁵, C(O)NR⁵C(O)N(R⁵)₂, P(O)(OR⁵)OH, P(O)(O)N(H)R⁵, P(O)(C(R⁶)₃)C(R⁶)₃, B(OH)₂, heterocyclyl or heteroaryl;
n is 0, 1, 2 or 3;
p is 0, 1, 2 or 3;
wherein any alkyl, alkylene, cycloalkyl, heterocyclyl, heteroaryl or aryl is optionally substituted with 1, 2 or 3 groups selected from OH, CN, SH, SCH₃, SO₂CH₃, SO₂NH₂, SO₂NH(C₁-C₄)alkyl, halogen, NH₂, NH(C₁-C₄)alkyl, N[(C₁-C₄)alkyl]₂, NH(aryl), C(O)NH₂, C(O)NH(alkyl), CH₂C(O)NH(alkyl), COOH, COOMe, acetyl, (C₁-C₈)alkyl, (C₁-C₈)haloalkyl, O(C₁-C₈)alkyl, O(C₁-C₈)haloalkyl, (C₂-C₈)alkenyl, (C₂-C₈)alkynyl, thioalkyl, cyanomethylene, alkylaminyl, alkylene-C(O)NH₂, alkylene-C(O)—NH(Me), NHC(O)alkyl, CH₂—C(O)—(C₁-C₈)alkyl, C(O)—(C₁-C₈)alkyl and alkylcarbonylaminyl, or a cycloalkyl, heterocyclyl, aryl or heteroaryl optionally substituted with OH, halogen, (C₁-C₈)alkyl, (C₁-C₈)haloalkyl, O(C₁-C₈)alkyl or O(C₁-C₈)haloalkyl,
wherein when X⁴ is a bond ring A forms a 5-membered heteroaryl wherein X¹, X⁵ and X⁶ can in addition to the above defined substituents be NR², and X¹ can in addition be —N-L¹-Y;
   wherein either Q is -L¹-Y, or X¹ is —C-L¹-Y or —N-L¹-Y; and
   wherein —X—R is covalently bonded to Formula I.

In certain embodiments, the present invention is directed to a translational inhibitor, wherein the translational inhibitor comprises an eIF4E Ligand having a structure encompassed by the genus of Formula I, or stereoisomers, tautomers or pharmaceutically acceptable salts thereof.

In other embodiments, the invention is directed to a translational inhibitor, wherein the translational inhibitor comprises an eIF4E Ligand having a structure according to Formula II:

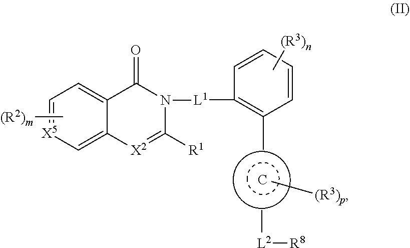

or stereoisomers, tautomers, or pharmaceutically acceptable salts thereof, wherein:
X² and X⁵ are independently CR² or N,
   or when X² is CR², R¹ and R² together with the atoms they attached to form a 6-membered aryl or heteroaryl;
L¹ is —(CH₂)—, —(CH₂)₂—, —(CH₂)₃—, —CH((C₁-C₈)alkyl)(CH₂)—, —CH((C₁-C₈)alkyl)(CH₂)₂—, —(CH₂)₂—O—, —CH₂CH═CH—, —CH₂C≡C— or —CH₂(cyclopropyl)-;
L² is —C(R⁶)(R⁶)—, —C(R⁶)(R⁶)C(R⁶)(R⁶)—, —C(R⁶)═C(R⁶)—, —N(R⁵)C(R⁶)(R⁶)—, —OC(R⁶)(R⁶)—, —C(═O)—, —C(═O)N(R⁵)C(R⁶)(R⁶)— or a bond;
Ring C is cycloalkyl, heterocyclyl, aryl or heteroaryl;
R¹ is H, OH, halo, CN, (C₁-C₈)alkyl, (C₁-C₈)haloalkyl, (C₃-C₆)cycloalkyl or NR⁵R⁵;
R² is independently H, halo, CN, NO, NO₂, C≡H, (C₁-C₈)alkyl, (C₁-C₈)haloalkyl, CH₂SR⁵, OR⁵, NHR⁵, NR⁵R⁵, [(C₁-C₈)alkylene]heterocyclyl, [(C₁-C₈)alkylene]heteroaryl, [(C₁-C₈)alkylene]NHR⁵, [(C₁-C₈)alkylene]NR⁵R⁵, [(C₁-C₈)alkylyne]NR⁵R⁵, C(O)R⁵, C(O)OR⁵, C(O)NHR⁵, C(O)NR⁵R⁵, SR⁵, S(O)R⁵, SO₂R⁵, SO₂NHR⁵, SO₂NR⁵R⁵, NH(CO)R⁶, NR⁵(CO)R⁶, aryl, heteroaryl, cycloalkyl or heterocyclyl;
R³ is independently OH, halo, CN, NO₂, (C₁-C₆)alkyl, (C₁-C₆)haloalkyl, (C₁-C₆)alkoxy, C≡H, NHR⁷, NR⁷R⁷, CO₂H, CO₂R⁷, [(C₁-C₃)alkylene](C₁-C₃)alkoxy, [(C₁-C₃)alkylene]CO₂H, (C₃-C₅)cycloalkyl, ═O. ═S, SR⁷, SO₂R⁷, NH(CO)R⁷ or NR⁷(CO)R⁷;
R⁵ is independently H, (C₁-C₃)alkyl, (C₁-C₃)haloalkyl, (C₃-C₅)cycloalkyl, CO₂H, [(C₁-C₃)alkylene]heteroaryl, [(C₁-C₃)alkylene]aryl, [(C₁-C₃)alkylene]CO₂H, heterocyclyl, aryl or heteroaryl,
   or wherein two R⁵ substituents together with a nitrogen atom form a 4-, 5-, 6-, or 7-membered heterocyclyl;
R⁶ is independently H, OH, halo, CN, (C₁-C₃)alkyl, (C₁-C₃)haloalkyl, (C₁-C₃)alkoxy, NHR⁷, NR⁷R⁷, CO₂H, [(C₁-C₃)alkylene]CO₂H, (C₃-C₅)cycloalkyl, SR⁷, NH(CO)R⁷ or NR⁷(CO)R⁷;
R⁷ is independently H, (C₁-C₈)alkyl, (C₁-C₈)haloalkyl, cycloalkyl, heterocyclyl, aryl or heteroaryl;
R⁸ is H, OH, CO₂H, CO₂R⁷, CF₂C(R⁶)₂OH, C(R⁶)₂OH, C(CF₃)₂OH, SO₂H, SO₃H, CF₂SO₂C(R⁶)₃, CF₂SO₂N(H)R⁵, SO₂N(H)R⁵, SO₂N(H)C(O)R⁶, C(O)N(H)SO₂R⁵, C(O)haloalkyl, C(O)N(H)OR⁵, C(O)N(R⁵)OH, C(O)N(H)R⁵, C(O)NR⁵C(O)N(R⁵)₂, P(O)(OR⁵)OH, P(O)(O)N(H)R⁵, P(O)(C(R⁶)₃)C(R⁶)₃, B(OH)₂, heterocyclyl or heteroaryl;
m is 0, 1, 2 or 3;
n is 0, 1, 2 or 3;
p is 0, 1, 2 or 3;
wherein any alkyl, alkylene, cycloalkyl, heterocyclyl, heteroaryl or aryl is optionally substituted with 1, 2 or 3 groups selected from OH, CN, SH, SCH₃, SO₂CH₃, SO₂NH₂, SO₂NH(C₁-C₄)alkyl, halogen, NH₂, NH(C₁-C₄)alkyl, N[(C₁-C₄)alkyl]₂, NH(aryl), C(O)NH₂, C(O)NH(alkyl), CH₂C(O)NH(alkyl), COOH, COOMe, acetyl, (C₁-C₈)alkyl, (C₁-C₈)haloalkyl, O(C₁-C₈)alkyl, O(C₁-C₈)haloalkyl, (C₂-C₈)alkenyl, (C₂-C₈)alkynyl, thioalkyl, cyanomethylene, alkylaminyl, alkylene-C(O)NH₂, alkylene-C(O)—NH(Me), NHC(O)alkyl, CH₂—C(O)—(C₁-C₈)alkyl, C(O)—(C₁-C₈)alkyl and alkylcarbonylaminyl, or a cycloalkyl, heterocyclyl, aryl or heteroaryl optionally substituted with OH, halogen, (C₁-C₈)alkyl, (C₁-C₈)haloalkyl, O(C₁-C₈)alkyl or O(C₁-C₈)haloalkyl; and
wherein —X—R is covalently bonded to Formula II.

In yet other embodiments, the invention is directed to a translational inhibitor, wherein the translational inhibitor comprises an eIF4E Ligand having a structure according to Formula III:

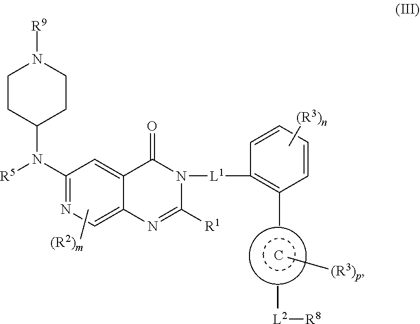

or stereoisomers, tautomers, or pharmaceutically acceptable salts thereof, wherein:
L¹ is —(CH₂)—, —(CH₂)₂—, —(CH₂)₃—, —CH((C₁-C₈)alkyl)(CH₂)—, —CH((C₁-C₈)alkyl)(CH₂)₂—, —(CH₂)₂—O—, —CH₂CH═CH—, —CH₂C≡C— or —CH₂(cyclopropyl)-;
L² is —C(R⁶)(R⁶)—, —C(R⁶)(R⁶)C(R⁶)(R⁶)—, —C(R⁶)═C(R⁶)—, —N(R⁵)C(R⁶)(R⁶)—, —OC(R⁶)(R⁶)—, —C(═O)—, —C(═O)N(R⁵)C(R⁶)(R⁶)— or a bond;
Ring C is a heteroaryl;
R¹ is H, OH, halo, CN, (C₁-C₈)alkyl, (C₁-C₈)haloalkyl, (C₃-C₆)cycloalkyl or NR⁵R⁵;
R² is independently H, halo, CN, NO, NO₂, C≡H, (C₁-C₈)alkyl, (C₁-C₈)haloalkyl, CH₂SR⁵, OR⁵, NHR⁵, NR⁵R⁵, [(C₁-C₈)alkylene]heterocyclyl, [(C₁-C₈)alkylene]heteroaryl, [(C₁-C₈)alkylene]NHR⁵, [(C₁-C₈)alkylene]NR⁵R⁵, [(C₁-C₈)alkylyne]NR⁵R⁵, C(O)R⁵, C(O)OR⁵, C(O)NHR⁵, C(O)NR⁵R⁵, SR⁵, S(O)R⁵, SO₂R⁵, SO₂NHR⁵, SO₂NR⁵R⁵, NH(CO)R⁶, NR⁵(CO)R⁶, aryl, heteroaryl, cycloalkyl or heterocyclyl;
R³ is independently OH, halo, CN, NO₂, (C₁-C₆)alkyl, (C₁-C₆)haloalkyl, (C₁-C₆)alkoxy, C≡H, NHR⁷, NR⁷R⁷, CO₂H, CO₂R⁷, [(C₁-C₃)alkylene](C₁-C₃)alkoxy, [(C₁-C₃)alkylene]CO₂H, (C₃-C₅)cycloalkyl, ═O. ═S, SR⁷, SO₂R⁷, NH(CO)R⁷ or NR⁷(CO)R⁷;
R⁵ is independently H, (C₁-C₃)alkyl, (C₁-C₃)haloalkyl, (C₃-C₅)cycloalkyl or heterocyclyl;
R⁶ is independently H, OH, halo, CN, (C₁-C₃)alkyl, (C₁-C₃)haloalkyl, (C₁-C₃)alkoxy, NHR⁷, NR⁷R⁷, CO₂H, [(C₁-C₃)alkylene]CO₂H, (C₃-C₅)cycloalkyl, SR⁷, NH(CO)R⁷ or NR⁷(CO)R⁷;
R⁷ is independently H, (C₁-C₈)alkyl, (C₁-C₈)haloalkyl, cycloalkyl, heterocyclyl, aryl or heteroaryl;
R⁸ is H, OH, CO₂H, CO₂R⁷, CF₂C(R⁶)₂OH, C(R⁶)₂OH, C(CF₃)₂OH, SO₂H, SO₃H, CF₂SO₂C(R⁶)₃, CF₂SO₂N(H)R⁵, SO₂N(H)R⁵, SO₂N(H)C(O)R⁶, C(O)N(H)SO₂R⁵, C(O)haloalkyl, C(O)N(H)OR⁵, C(O)N(R⁵)OH, C(O)N(H)R⁵, C(O)NR⁵C(O)N(R⁵)₂, P(O)(OR⁵)OH, P(O)(O)N(H)R⁵, P(O)(C(R⁶)₃)C(R⁶)₃, B(OH)₂, heterocyclyl or heteroaryl;
R⁹ is H, (C₁-C₈)alkyl, (C₁-C₈)haloalkyl, cycloalkyl or heterocyclyl;
m is 0, 1, or 2;
n is 0, 1, 2 or 3;
p is 0, 1, 2 or 3;
wherein any alkyl, alkylene, cycloalkyl, heterocyclyl, heteroaryl or aryl is optionally substituted with 1, 2 or 3 groups selected from OH, CN, SH, SCH₃, SO₂CH₃, SO₂NH₂, SO₂NH(C₁-C₄)alkyl, halogen, NH₂, NH(C₁-C₄)alkyl, N[(C₁-C₄)alkyl]₂, NH(aryl), C(O)NH₂, C(O)NH(alkyl), CH₂C(O)NH(alkyl), COOH, COOMe, acetyl, (C₁-C₈)alkyl, (C₁-C₈)haloalkyl, O(C₁-C₈)alkyl, O(C₁-C₈)haloalkyl, (C₂-C₈)alkenyl, (C₂-C₈)alkynyl, thioalkyl, cyanomethylene, alkylaminyl, alkylene-C(O)NH₂, alkylene-C(O)—NH(Me), NHC(O)alkyl, CH₂—C(O)—(C₁-C₈)alkyl, C(O)—(C₁-C₈)alkyl and alkylcarbonylaminyl, and
wherein —X—R is covalently bonded to Formula III.

In still other embodiments, the invention is directed to a translational inhibitor, wherein the translational inhibitor comprises an eIF4E Ligand having a structure according to Formula IV:

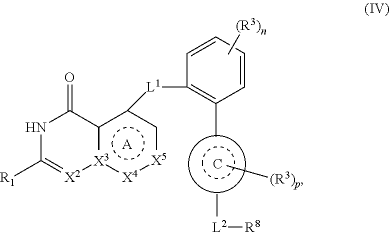

or stereoisomers, tautomers, or pharmaceutically acceptable salts thereof, wherein:
X² and X⁵ are independently CR² or N,
   or when X² is CR², R¹ and R² together with the atoms they attached to form a 6-membered aryl or heteroaryl;
X³ is C, or X³ is C or N when X⁴ is a bond;
X⁴ is a bond, CR² or N,
   wherein X⁴ and X⁵ together with 3 or 4 carbon or nitrogen atoms combine to form a 5- or 6-membered heteroaryl;
L¹ is —(CH₂)—, —(CH₂)₂—, —(CH₂)₃—, —CH((C₁-C₈)alkyl)(CH₂)—, —CH((C₁-C₈)alkyl)(CH₂)₂—, —(CH₂)₂—O—, —CH₂CH═CH—, —CH₂C≡C— or —CH₂(cyclopropyl)-;
L² is —C(R⁶)(R⁶)—, —C(R⁶)(R⁶)C(R⁶)(R⁶)—, —C(R⁶)═C(R⁶)—, —N(R⁵)C(R⁶)(R⁶)—, —OC(R⁶)(R⁶)—, —C(═O)—, —C(═O)N(R⁵)C(R⁶)(R⁶)—;
Ring C is cycloalkyl, heterocyclyl, aryl or heteroaryl;
R¹ is H, OH, halo, CN, (C₁-C₈)alkyl, (C₁-C₈)haloalkyl, (C₃-C₆)cycloalkyl or NR⁵R⁵;
R² is independently H, halo, CN, NO, NO₂, C≡H, (C₁-C₈)alkyl, (C₁-C₈)haloalkyl, CH₂SR⁵, OR⁵, NHR⁵, NR⁵R⁵, [(C₁-C₈)alkylene]heterocyclyl, [(C₁-C₈)alkylene]heteroaryl, [(C₁-C₈)alkylene]NHR⁵, [(C₁-C₈)alkylene]NR⁵R⁵, [(C₁-C₈)alkylyne]NR⁵R⁵, C(O)R⁵, C(O)OR⁵, C(O)NHR⁵, C(O)NR⁵R⁵, SR⁵, S(O)R⁵, SO₂R⁵, SO₂NHR⁵, SO₂NR⁵R⁵, NH(CO)R⁶, NR⁵(CO)R⁶, aryl, heteroaryl, cycloalkyl or heterocyclyl;
R³ is independently OH, halo, CN, NO₂, (C₁-C₆)alkyl, (C₁-C₆)haloalkyl, (C₁-C₆)alkoxy, C≡H, NHR⁷, NR⁷R⁷, CO₂H, CO₂R⁷, [(C₁-C₃)alkylene](C₁-C₃)alkoxy, [(C₁-C₃)alkylene]CO₂H, (C₃-C₅)cycloalkyl, ═O. ═S, SR⁷, SO₂R⁷, NH(CO)R⁷ or NR⁷(CO)R⁷;
R⁵ is independently H, (C₁-C₃)alkyl, (C₁-C₃)haloalkyl, (C₃-C₅)cycloalkyl, CO₂H, [(C₁-C₃)alkylene]heteroaryl, [(C₁-C₃)alkylene]aryl, [(C₁-C₃)alkylene]CO₂H, heterocyclyl, aryl or heteroaryl,
   or wherein two R⁵ substituents together with a nitrogen atom form a 4-, 5-, 6- or 7-membered heterocyclyl;
R⁶ is independently H, OH, halo, CN, (C₁-C₃)alkyl, (C₁-C₃)haloalkyl, (C₁-C₃)alkoxy, NHR⁷, NR⁷R⁷, CO₂H, [(C₁-C₃)alkylene]CO₂H, (C₃-C₅)cycloalkyl, SR⁷, NH(CO)R⁷ or NR⁷(CO)R⁷;
R⁷ is independently H, (C₁-C₈)alkyl, (C₁-C₈)haloalkyl, cycloalkyl, heterocyclyl, aryl or heteroaryl;
R⁸ is H, OH, CO₂H, CO₂R⁷, CF₂C(R⁶)₂OH, C(R⁶)₂OH, C(CF₃)₂OH, SO₂H, SO₃H, CF₂SO₂C(R⁶)₃, CF₂SO₂N(H)R⁵, SO₂N(H)R⁵, SO₂N(H)C(O)R⁶, C(O)N(H)SO₂R⁵, C(O)haloalkyl, C(O)N(H)OR⁵, C(O)N(R⁵)OH, C(O)N(H)R⁵, C(O)NR⁵C(O)N(R⁵)₂, P(O)(OR⁵)OH, P(O)(O)N(H)R⁵, P(O)(C(R⁶)₃)C(R⁶)₃, B(OH)₂, heterocyclyl or heteroaryl;
n is 0, 1, 2 or 3;
p is 0, 1, 2 or 3;
wherein any alkyl, alkylene, cycloalkyl, heterocyclyl, heteroaryl or aryl is optionally substituted with 1, 2 or 3 groups selected from OH, CN, SH, SCH₃, SO₂CH₃, SO₂NH₂, SO₂NH(C₁-C₄)alkyl, halogen, NH₂, NH(C₁-C₄)alkyl, N[(C₁-C₄)alkyl]₂, NH(aryl), C(O)NH₂, C(O)NH(alkyl), CH₂C(O)NH(alkyl), COOH, COOMe, acetyl, (C₁-C₈)alkyl, (C₁-C₈)haloalkyl, O(C₁-C₈)alkyl, O(C₁-C₈)haloalkyl, (C₂-C₈)alkenyl, (C₂-C₈)alkynyl, thioalkyl, cyanomethylene, alkylaminyl, alkylene-C(O)NH₂, alkylene-C(O)—NH(Me), NHC(O)alkyl, CH₂—C(O)—(C₁-C₈)alkyl, C(O)—(C₁-C₈)alkyl and alkylcarbonylaminyl, or a cycloalkyl, heterocyclyl, aryl or heteroaryl optionally substituted with OH, halogen, (C₁-C₈)alkyl, (C₁-C₈)haloalkyl, O(C₁-C₈)alkyl or O(C₁-C₈)haloalkyl,
wherein when X⁴ is a bond, ring A forms a 5-membered heteroaryl wherein X¹ and X⁵ can in addition to C be N; and
wherein —X—R is covalently bonded to Formula IV.

In additional embodiments, the invention is directed to a translational inhibitor, wherein the translational inhibitor comprises an eIF4E Ligand having a structure according to Formula V:

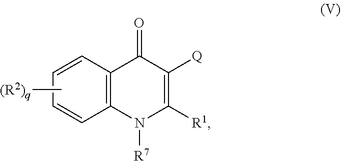

or stereoisomers, tautomers, or pharmaceutically acceptable salts thereof, wherein:
Q is -L¹-Y;
L¹ is —(CH₂)—, —(CH₂)₂—, —(CH₂)₃—, —CH((C₁-C₈)alkyl)(CH₂)—, —CH((C₁-C₈)alkyl)(CH₂)₂—, —(CH₂)₂—O—, —CH₂CH═CH—, —CH₂C≡C— or —CH₂(cyclopropyl)-;
Y is

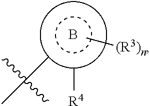

wherein
Ring B is a six-membered aryl, heteroaryl or heterocyclyl;
R¹ is H, OH, halo, CN, (C₁-C₈)alkyl, (C₁-C₈)haloalkyl, (C₃-C₆)cycloalkyl or NR⁵R⁵;
R² is independently H, halo, CN, NO, NO₂, C≡H, (C₁-C₈)alkyl, (C₁-C₈)haloalkyl, CH₂SR⁵, OR⁵, NHR⁵, NR⁵R⁵, [(C₁-C₈)alkylene]heterocyclyl, [(C₁-C₈)alkylene]heteroaryl, [(C₁-C₈)alkylene]NHR⁵, [(C₁-C₈)alkylene]NR⁵R⁵, [(C₁-C₈)alkylyne]NR⁵R⁵, C(O)R⁵, C(O)OR⁵, C(O)NHR⁵, C(O)NR⁵R⁵, SR⁵, S(O)R⁵, SO₂R⁵, SO₂NHR⁵, SO₂NR⁵R⁵, NH(CO)R⁶, NR⁵(CO)R⁶, aryl, heteroaryl, cycloalkyl or heterocyclyl;
R³ is independently OH, halo, CN, NO₂, (C₁-C₆)alkyl, (C₁-C₆)haloalkyl, (C₁-C₆)alkoxy, C—H, NHR⁷, NR⁷R⁷, CO₂H, CO₂R⁷, [(C₁-C₃)alkylene](C₁-C₃)alkoxy, [(C₁-C₃)alkylene]CO₂H, (C₃-C₅)cycloalkyl, ═O. ═S, SR⁷, SO₂R⁷, NH(CO)R⁷ or NR⁷(CO)R⁷;
R⁴ is H, OH, halo, CN, (C₁-C₃)alkyl, (C₁-C₃)haloalkyl, (C₁-C₃)alkoxy, SR⁷ or Z, wherein Z is

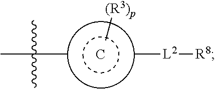

Ring C is cycloalkyl, heterocyclyl, aryl or heteroaryl;
L² is —C(R⁶)(R⁶)—, —C(R⁶)(R⁶)C(R⁶)(R⁶)—, —C(R⁶)═C(R⁶)—, —N(R⁵)C(R⁶)(R⁶)—, —OC(R⁶)(R⁶)—, —C(═O)—, —C(═O)N(R⁵)C(R⁶)(R⁶)— or a bond;
R⁵ is independently H, (C₁-C₃)alkyl, (C₁-C₃)haloalkyl, (C₃-C₅)cycloalkyl, CO₂H, [(C₁-C₃)alkylene]heteroaryl, [(C₁-C₃)alkylene]aryl, [(C₁-C₃)alkylene]CO₂H, heterocyclyl, aryl or heteroaryl,
   or wherein two R⁵ substituents together with a nitrogen atom form a 4-, 5-, or 6-membered heterocyclyl;
R⁶ is independently H, OH, halo, CN, (C₁-C₃)alkyl, (C₁-C₃)haloalkyl, (C₁-C₃)alkoxy, NHR⁷, NR⁷R⁷, CO₂H, [(C₁-C₃)alkylene]CO₂H, (C₃-C₅)cycloalkyl, SR⁷, NH(CO)R⁷ or NR⁷(CO)R⁷;
R⁷ is independently H, (C₁-C₈)alkyl, (C₁-C₈)haloalkyl, cycloalkyl, heterocyclyl, aryl or heteroaryl;
R⁸ is H, OH, CO₂H, CO₂R⁷, CF₂C(R⁶)₂OH, C(R⁶)₂OH, C(CF₃)₂OH, SO₂H, SO₃H, CF₂SO₂C(R⁶)₃, CF₂SO₂N(H)R⁵, SO₂N(H)R⁵, SO₂N(H)C(O)R⁶, C(O)N(H)SO₂R⁵, C(O)haloalkyl, C(O)N(H)OR⁵, C(O)N(R⁵)OH, C(O)N(H)R⁵, P(O)(OR⁵)OH, P(O)(O)N(H)R⁵, P(O)(C(R⁶)₃)C(R⁶)₃, B(OH)₂, heterocyclyl or heteroaryl;
n is 0, 1, 2 or 3;
p is 0, 1, 2 or 3;
q is 0, 1, 2, 3 or 4;
wherein any alkyl, alkylene, cycloalkyl, heterocyclyl, heteroaryl or aryl is optionally substituted with 1, 2 or 3 groups selected from OH, CN, SH, SCH₃, SO₂CH₃, SO₂NH₂, SO₂NH(C₁-C₄)alkyl, halogen, NH₂, NH(C₁-C₄)alkyl, N[(C₁-C₄)alkyl]₂, NH(aryl), C(O)NH₂, C(O)NH(alkyl), CH₂C(O)NH(alkyl), COOH, COOMe, acetyl, (C₁-C₈)alkyl, (C₁-C₈)haloalkyl, O(C₁-C₈)alkyl, O(C₁-C₈)haloalkyl, (C₂-C₈)alkenyl, (C₂-C₈)alkynyl, thioalkyl, cyanomethylene, alkylaminyl, alkylene-C(O)NH₂, alkylene-C(O)—NH(Me), NHC(O)alkyl, CH₂—C(O)—(C₁-C₈)alkyl, C(O)—(C₁-C₈)alkyl and alkylcarbonylaminyl, or a cycloalkyl, heterocyclyl, aryl or heteroaryl optionally substituted with OH, halogen, (C₁-C₈)alkyl, (C₁-C₈)haloalkyl, O(C₁-C₈)alkyl or O(C₁-C₈)haloalkyl, and
wherein —X—R is covalently bonded to Formula V.

In additional embodiments, the invention is directed to a translational inhibitor, wherein the translational inhibitor comprises an eIF4E Ligand having a structure according to Formula VI:

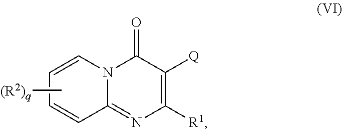

or stereoisomers, tautomers, or pharmaceutically acceptable salts thereof, wherein:
Q is -L¹-Y;
L¹ is —(CH₂)—, —(CH₂)₂—, —(CH₂)₃—, —CH((C₁-C₈)alkyl)(CH₂)—, —CH((C₁-C₈)alkyl)(CH₂)₂—, —(CH₂)₂—O—, —CH₂CH═CH—, —CH₂C≡C— or —CH₂(cyclopropyl)-;
Y is

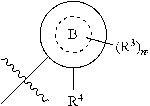

wherein
Ring B is a six-membered aryl, heteroaryl or heterocyclyl;
R¹ is H, OH, halo, CN, (C₁-C₈)alkyl, (C₁-C₈)haloalkyl, (C₃-C₆)cycloalkyl or NR⁵R⁵;
R² is independently H, halo, CN, NO, NO₂, C≡H, (C₁-C₈)alkyl, (C₁-C₈)haloalkyl, CH₂SR⁵, OR⁵, NHR⁵, NR⁵R⁵, [(C₁-C₈)alkylene]heterocyclyl, [(C₁-C₈)alkylene]heteroaryl, [(C₁-C₈)alkylene]NHR⁵, [(C₁-C₈)alkylene]NR⁵R⁵, [(C₁-C₈)alkylyne]NR⁵R⁵, C(O)R⁵, C(O)OR⁵, C(O)NHR⁵, C(O)NR⁵R⁵, SR⁵, S(O)R⁵, SO₂R⁵, SO₂NHR⁵, SO₂NR⁵R⁵, NH(CO)R⁶, NR⁵(CO)R⁶, aryl, heteroaryl, cycloalkyl or heterocyclyl;
R³ is independently OH, halo, CN, NO₂, (C₁-C₆)alkyl, (C₁-C₆)haloalkyl, (C₁-C₆)alkoxy, C—H, NHR⁷, NR⁷R⁷, CO₂H, CO₂R⁷, [(C₁-C₃)alkylene](C₁-C₃)alkoxy, [(C₁-C₃)alkylene]CO₂H, (C₃-C₅)cycloalkyl, ═O. ═S, SR⁷, SO₂R⁷, NH(CO)R⁷ or NR⁷(CO)R⁷;
R⁴ is H, OH, halo, CN, (C₁-C₃)alkyl, (C₁-C₃)haloalkyl, (C₁-C₃)alkoxy, SR⁷ or Z, wherein Z is

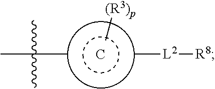

Ring C is cycloalkyl, heterocyclyl, aryl or heteroaryl;
L² is —C(R⁶)(R⁶)—, —C(R⁶)(R⁶)C(R⁶)(R⁶)—, —C(R⁶)═C(R⁶)—, —N(R⁵)C(R⁶)(R⁶)—, —OC(R⁶)(R⁶)—, —C(═O)—, —C(═O)N(R⁵)C(R⁶)(R⁶)— or a bond;
R⁵ is independently H, (C₁-C₃)alkyl, (C₁-C₃)haloalkyl, (C₃-C₅)cycloalkyl, CO₂H, [(C₁-C₃)alkylene]heteroaryl, [(C₁-C₃)alkylene]aryl, [(C₁-C₃)alkylene]CO₂H, heterocyclyl, aryl or heteroaryl,
   or wherein two R⁵ substituents together with a nitrogen atom form a 4-, 5-, or 6-membered heterocyclyl;
R⁶ is independently H, OH, halo, CN, (C₁-C₃)alkyl, (C₁-C₃)haloalkyl, (C₁-C₃)alkoxy, NHR⁷, NR⁷R⁷, CO₂H, [(C₁-C₃)alkylene]CO₂H, (C₃-C₅)cycloalkyl, SR⁷, NH(CO)R⁷ or NR⁷(CO)R⁷;
R⁷ is independently H, (C₁-C₈)alkyl, (C₁-C₈)haloalkyl, cycloalkyl, heterocyclyl, aryl or heteroaryl;
R⁸ is H, OH, CO₂H, CO₂R⁷, CF₂C(R⁶)₂OH, C(R⁶)₂OH, C(CF₃)₂OH, SO₂H, SO₃H, CF₂SO₂C(R⁶)₃, CF₂SO₂N(H)R⁵, SO₂N(H)R⁵, SO₂N(H)C(O)R⁶, C(O)N(H)SO₂R⁵, C(O)haloalkyl, C(O)N(H)OR⁵, C(O)N(R⁵)OH, C(O)N(H)R⁵, C(O)NR⁵C(O)N(R⁵)₂, P(O)(OR⁵)OH, P(O)(O)N(H)R⁵, P(O)(C(R⁶)₃)C(R⁶)₃, B(OH)₂, heterocyclyl or heteroaryl;
n is 0, 1, 2 or 3;
p is 0, 1, 2 or 3;
q is 0, 1, 2, 3 or 4;
wherein any alkyl, alkylene, cycloalkyl, heterocyclyl, heteroaryl or aryl is optionally substituted with 1, 2 or 3 groups selected from OH, CN, SH, SCH₃, SO₂CH₃, SO₂NH₂, SO₂NH(C₁-C₄)alkyl, halogen, NH₂, NH(C₁-C₄)alkyl, N[(C₁-C₄)alkyl]₂, NH(aryl), C(O)NH₂, C(O)NH(alkyl), CH₂C(O)NH(alkyl), COOH, COOMe, acetyl, (C₁-C₈)alkyl, (C₁-C₈)haloalkyl, O(C₁-C₈)alkyl, O(C₁-C₈)haloalkyl, (C₂-C₈)alkenyl, (C₂-C₈)alkynyl, thioalkyl, cyanomethylene, alkylaminyl, alkylene-C(O)NH₂, alkylene-C(O)—NH(Me), NHC(O)alkyl, CH₂—C(O)—(C₁-C₈)alkyl, C(O)—(C₁-C₈)alkyl and alkylcarbonylaminyl, or a cycloalkyl, heterocyclyl, aryl or heteroaryl optionally substituted with OH, halogen, (C₁-C₈)alkyl, (C₁-C₈)haloalkyl, O(C₁-C₈)alkyl or O(C₁-C₈)haloalkyl, and
wherein —X—R is covalently bonded to Formula VI.

In one embodiment X² of Formulae I, II, and IV is N.

In one embodiment X³ of Formulae I and IV is C.

In one embodiment X⁴ of Formulae I and IV is CR² or N.

In one embodiment X⁵ of Formulae I and IV is CR².

In one embodiment L¹ of Formulae I, II, III, IV, V and VI is —(CH₂)₂—O—, —CH₂CH═CH— or —CH₂C≡C—. In another embodiment L¹ is —(CH₂)₂—O—.

In one embodiment L² of Formulae I, II, III, IV, V and VI is a bond.

In one embodiment Ring B of Formulae I, V and VI is aryl.

In one embodiment Ring C of Formulae I, II, III, IV, V and VI is heteroaryl.

In one embodiment Ring C of Formulae I, II, III, IV, V and VI is

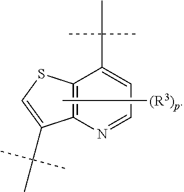

In one embodiment Ring C of Formula III is

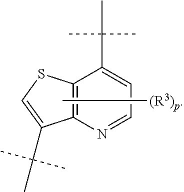

In one embodiment R¹ of Formulae I, II, III, IV, V and VI is H, (C₁-C₈)alkyl or (C₁-C₈)haloalkyl.

In one embodiment R¹ of Formula IV is NHR⁵ or N[(C₁-C₃)alkyl](R⁵).

In one embodiment R² of Formulae I, II, III, IV, V and VI is halo, CN, (C₁-C₈)alkyl, (C₁-C₈)haloalkyl or OR⁵. In another embodiment R² is halo, CN or (C₁-C₈)haloalkyl.

In one embodiment R³ of Formulae I, II, III, IV, V and VI is halo, CN, (C₁-C₃)alkyl or (C₁-C₃)haloalkyl.

In one embodiment R⁴ of Formulae I, V and VI is Z, wherein Z is

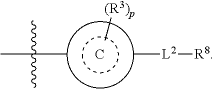

In one embodiment R⁵ of Formulae I, II, III, V and VI is H, (C₁-C₃)alkyl or (C₁-C₃)haloalkyl. In another embodiment R⁵ of Formula IV is aryl.

In one embodiment R⁶ of Formulae I, II, III, IV, V and VI is H, OH, halo, CN, (C₁-C₃)alkyl, (C₁-C₃)haloalkyl or (C₁-C₃)alkoxy.

In one embodiment R⁷ of Formulae I, II, III, IV, V and VI is H, (C₁-C₈)alkyl or (C₁-C₈)haloalkyl.

In one embodiment R⁸ of Formulae I, II, III, IV, V and VI is CO₂H or C(O)N(H)SO₂R⁵.

In one embodiment R⁹ of Formula III is (C₁-C₈)alkyl or (C₁-C₈)haloalkyl.

In one embodiment R⁹ of Formula III is cycloalkyl or heterocyclyl.

In one embodiment “m” of Formulae I and II=2 or 3. In another embodiment “n” of Formulae I, II, IV, V and VI=1 or 2. In yet another embodiment “p” of Formulae I, II, III, IV, V and VI=0 or 1.

In one embodiment the optional substituents of alkyl, cycloalkyl, heterocyclyl, heteroaryl or aryl are OH, CN, halogen, (C₁-C₈)alkyl, O(C₁-C₈)alkyl, haloalkyl, alkylene-C(O)NH₂ or alkylene-C(O)—NH(Me).

In one embodiment the optional substituents of alkyl, cycloalkyl, heterocyclyl, heteroaryl or aryl are cycloalkyl, heterocyclyl, aryl or heteroaryl optionally substituted with OH, halogen, (C₁-C₈)alkyl, (C₁-C₈)haloalkyl, O(C₁-C₈)alkyl or O(C₁-C₈)haloalkyl.

In one embodiment —X—R is covalently bonded to R⁸ of Formulae I, II, III, IV, V or VI of the eIF4E Ligand.

The inventive compounds according to Formulae I, II, III, IV, V and VI may be isotopically-labeled by having one or more atoms replaced by an atom having a different atomic mass or mass number. Examples of isotopes that can be incorporated into compounds according to Formulae I, II, III, IV, V and VI include isotopes of hydrogen, carbon, nitrogen, oxygen, phosphorous, fluorine, chlorine, or iodine. Illustrative of such isotopes are ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I respectively. These radiolabeled compounds can be used to measure the biodistribution, tissue concentration and the kinetics of transport and excretion from biological tissues including a subject to which such a labeled compound is administered. Labeled compounds are also used to determine therapeutic effectiveness, the site or mode of action, and the binding affinity of a candidate therapeutic to a pharmacologically important target. Certain radioactive-labeled compounds according to Formulae I, II, III, IV, V and VI, therefore, are useful in drug and/or tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, affords certain therapeutic advantages resulting from the greater metabolic stability, for example, increased in vivo half-life of compounds containing deuterium. Substitution of hydrogen with deuterium may reduce dose required for therapeutic effect, and hence may be preferred in a discovery or clinical setting.

Substitution with positron emitting isotopes, such as ¹¹C, ¹⁸F, ¹⁵O and ¹³N, provides labeled analogs of the inventive compounds that are useful in Positron Emission Tomography (PET) studies, e.g., for examining substrate receptor occupancy. Isotopically-labeled compounds according to Formulae I, II, III, IV, V and VI can generally be prepared by conventional techniques known to those skilled in the art or by processes analogous to those described in the Preparations and Examples section as set out below using an appropriate isotopic-labeling reagent.

Embodiments of the invention disclosed herein are also meant to encompass the in vivo metabolic products of compounds according to Formulae I, II, III, IV, V and VI. Such products may result from, for example, the oxidation, reduction, hydrolysis, amidation, esterification, and like processes primarily due to enzymatic activity upon administration of a compound of the invention. Accordingly, the invention includes compounds that are produced as by-products of enzymatic or non-enzymatic activity on an inventive compound following the administration of such a compound to a mammal for a period of time sufficient to yield a metabolic product. Metabolic products, particularly pharmaceutically active metabolites are typically identified by administering a radiolabeled compound of the invention in a detectable dose to a subject, such as rat, mouse, guinea pig, monkey, or human, for a sufficient period of time during which metabolism occurs, and isolating the metabolic products from urine, blood or other biological samples that are obtained from the subject receiving the radiolabeled compound.

The invention also provides pharmaceutically acceptable salt forms of compounds in Formulae I, II, III, IV, V and VI. Encompassed within the scope of the invention are both acid and base addition salts that are formed by contacting a pharmaceutically suitable acid or a pharmaceutically suitable base with a compound of the invention.

In certain embodiments, the present invention provides a translational inhibitor comprising an eIF4E ligand, wherein the eIF4E ligand has a structure according to Formula I, II, III, IV, V, or VI. In some embodiments, the eIF4E ligand of Formula I, II, III, IV, V, or VI is attached directly to a chemical moiety that binds covalently with eIF4E. In other embodiments, the eIF4E ligand of Formula I, II, III, IV, V, or VI is attached via a linker to a chemical moiety that binds covalently with eIF4E. In certain aspects, covalent binding of the chemical moiety with eIF4E is reversible. In other aspects, covalent binding of the chemical moiety with eIF4E is irreversible.

In one embodiment the translational inhibitor of the invention is

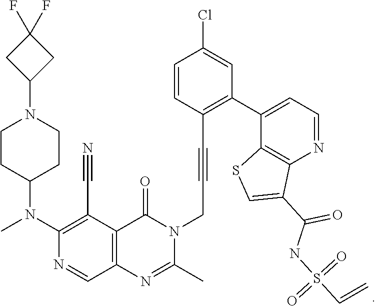 [SMILES: C=CS(=O)(=O)NC(=O)c1csc2c(-c3cc(Cl)ccc3C#CCn3c(C)nc4cnc(N(C)C5CCN(C6CC(F)(F)C6)CC5)c(C#N)c4c3=O)ccnc12]

In another embodiment the translational inhibitor of the invention is

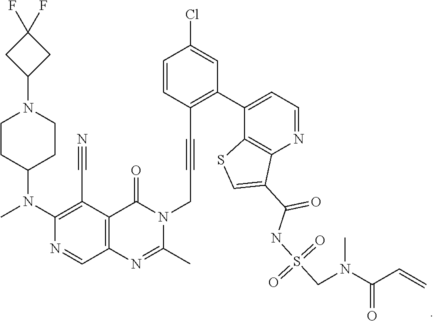 [SMILES: C=CC(=O)N(C)CS(=O)(=O)NC(=O)c1csc2c(-c3cc(Cl)ccc3C#CCn3c(C)nc4cnc(N(C)C5CCN(C6CC(F)(F)C6)CC5)c(C#N)c4c3=O)ccnc12]

In yet another embodiment the translational inhibitor of the invention is

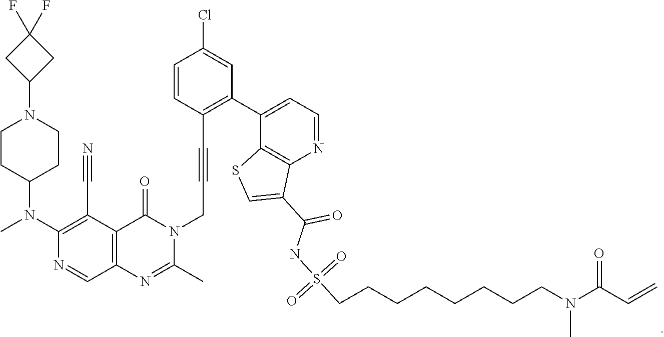 [SMILES: C=CC(=O)N(C)CCCCCCCCS(=O)(=O)NC(=O)c1csc2c(-c3cc(Cl)ccc3C#CCn3c(C)nc4cnc(N(C)C5CCN(C6CC(F)(F)C6)CC5)c(C#N)c4c3=O)ccnc12]

In still another embodiment the translational inhibitor of the invention is

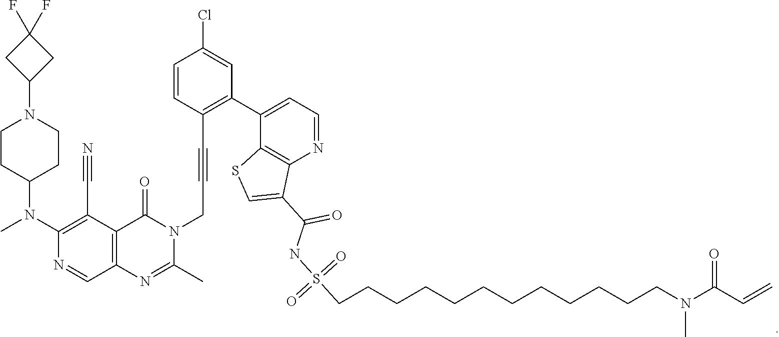 [SMILES: C=CC(=O)N(C)CCCCCCCCCCCCS(=O)(=O)NC(=O)c1csc2c(-c3cc(Cl)ccc3C#CCn3c(C)nc4cnc(N(C)C5CCN(C6CC(F)(F)C6)CC5)c(C#N)c4c3=O)ccnc12]

In an additional embodiment the translational inhibitor of the invention is

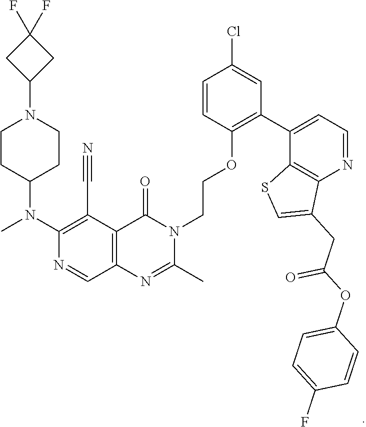 [SMILES: Cc1nc2cnc(N(C)C3CCN(C4CC(F)(F)C4)CC3)c(C#N)c2c(=O)n1CCOc1ccc(Cl)cc1-c1ccnc2c(CC(=O)Oc3ccc(F)cc3)csc12]

In yet an additional embodiment the translational inhibitor is

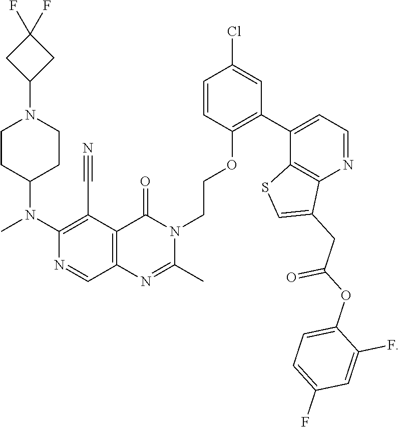 [SMILES: Cc1nc2cnc(N(C)C3CCN(C4CC(F)(F)C4)CC3)c(C#N)c2c(=O)n1CCOc1ccc(Cl)cc1-c1ccnc2c(CC(=O)Oc3ccc(F)cc3F)csc12]

### Preparation of the Translational Inhibitors

The eIF4E Ligands of the invention having a structure according to Formula I, II, III, IV, V, or VI may be synthesized as described in U.S. Provisional Application No. 62/869,662, which is incorporated herein by reference in its entirety.

The translational inhibitors of the invention may be synthesized using standard synthetic techniques known to those of skill in the art or using methods known in the art in combination with methods described herein. In addition, solvents, temperatures and other reaction conditions presented herein may vary according to those of skill in the art. As a further guide the following synthetic methods may also be utilized.

The reactions can be employed in a linear sequence to provide the compounds described herein or they may be used to synthesize fragments which are subsequently joined by the methods described herein and/or known in the art.

### Pharmaceutical Compositions

In certain embodiments, the present invention provides pharmaceutical compositions comprising (i) a therapeutically effective amount of the translational inhibitor of the invention, or a stereoisomer, a tautomer or a pharmaceutically acceptable salt thereof; and (ii) one or more pharmaceutically acceptable carriers, diluents or excipients.

In some embodiments, pharmaceutical compositions optionally comprise one or more additional active substances, e.g., therapeutically and/or prophylactically active substances. In certain cases, pharmaceutical compositions of the present invention are sterile and/or pyrogen-free. General considerations in the formulation and/or manufacture of pharmaceutical agents may be found, for example, in Remington: The Science and Practice of Pharmacy 21st ed., Lippincott Williams & Wilkins, 2005 (incorporated herein by reference in its entirety).

Although the descriptions of pharmaceutical compositions provided herein are principally directed to pharmaceutical compositions which are suitable for administration to humans, it will be understood by the skilled artisan that such compositions are generally suitable for administration to animals of all sorts.

In specific embodiments, a translational inhibitor of the invention is formulated as a pharmaceutical composition in an amount effective to treat a particular disease or condition of interest (e.g., cancer, cardiovascular disease, or autoimmune disease) upon administration of the pharmaceutical composition to a subject. In certain aspects, the subject is a mammal. In certain aspects, the mammal is a human.

A “mammal” includes primates, such as humans, monkeys and apes, and non-primates such as domestic animals, including laboratory animals and household pets (e.g., cats, dogs, swine, cattle, sheep, goats, horses, rabbits), and non-domestic animals, such as wildlife or the like.

In particular embodiments, a pharmaceutical composition comprises a translational inhibitor of the invention and a pharmaceutically acceptable carrier, diluent or excipient. In this regard, a “pharmaceutically acceptable carrier, diluent or excipient” includes any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier that has been approved by the United States Food and Drug Administration as being acceptable for use in humans or domestic animals.

In certain cases, a pharmaceutical composition of the invention is prepared by combining or formulating a translational inhibitor of the invention with an appropriate pharmaceutically acceptable carrier, diluent or excipient. In certain aspects, a pharmaceutical composition of the invention is formulated into preparations in solid, semi-solid, liquid or gaseous forms, including, but not limited to, tablets, capsules, powders, granules, ointments, solutions, suppositories, injections, inhalants, gels, microspheres, and aerosols.

Exemplary routes of administering such pharmaceutical compositions include oral, topical, transdermal, inhalation, parenteral, sublingual, buccal, rectal, vaginal, and intranasal. Thus, in certain embodiments, a translational inhibitor or a pharmaceutical composition of the invention is formulated to be administered by routes selected from the group consisting of oral, topical, transdermal, inhalation, parenteral, sublingual, buccal, rectal, vaginal, and intranasal routes. In specific embodiments, the translational inhibitors or pharmaceutical compositions of the invention are administered orally. The term parenteral, as used herein, includes subcutaneous injections, intravenous, intramuscular, intrasternal injection or infusion techniques. In certain aspects, the translational inhibitors or pharmaceutical compositions of the invention are formulated to allow the active ingredients contained therein to be bioavailable upon administration to a subject or patient.

In certain aspects, pharmaceutical compositions that will be administered to a subject or patient take the form of one or more dosage units, where, for example, a tablet may be a single dosage unit, and a container of a translational inhibitor of the invention in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). A composition to be administered will, in any event, contain a therapeutically effective amount of a translational inhibitor of the invention, or a pharmaceutically acceptable salt thereof, to aid in treatment of a disease or condition of interest in accordance with the teachings herein.

In certain embodiments, the pharmaceutical compositions of the invention are used to treat a disease including, but not limited to, hyperproliferative disease, autoimmune disease, diabetes, neurodegenerative disease, inflammatory disease, viral infection, cardiovascular disease, metabolic disease, genetic disease such as Alzheimer's, Parkinson's, Fragile X Syndrome and autism, or any combination thereof.

In some aspects, the disease is a hyperproliferative disease. In specific embodiments, the hyperproliferative disease is a cancer. In certain embodiments, the cancer includes, without limitation, solid tumor, melanoma, multiple melanoma, non-small cell lung cancer, renal cell carcinoma, renal cancer, a hematological cancer, prostate cancer, castration-resistant prostate cancer, colon cancer, rectal cancer, gastric cancer, esophageal cancer, bladder cancer, head and neck cancer, thyroid cancer, breast cancer, triple-negative breast cancer, ovarian cancer, cervical cancer, lung cancer, urothelial cancer, pancreatic cancer, glioblastoma, hepatocellular cancer, myeloma, multiple myeloma, leukemia, B-cell lymphoma, T-cell lymphoma, hairy cell lymphoma, Hodgkin's lymphoma, non-Hodgkin's lymphoma, Burkitt's lymphoma, myelodysplastic syndrome, brain cancer, CNS cancer, malignant glioma, or any combination thereof.

In certain instances, a pharmaceutical composition of a translational inhibitor of the invention may be in the form of a solid or liquid. In some aspects, the carrier(s) are particulate so that the compositions are, for example, in tablet or powder form. In other aspects, the carrier(s) are liquid, with a composition being, for example, an oral syrup, injectable liquid or an aerosol, which is useful in, for example, inhalatory administration. When intended for oral administration, a pharmaceutical composition of a translational inhibitor of the invention is preferably in either solid or liquid form, where semi-solid, semi-liquid, suspension and gel forms are included within the forms considered herein as either solid or liquid.

In certain aspects, as a solid composition for oral administration, a pharmaceutical composition of a translational inhibitor of the invention may be formulated into a powder, granule, compressed tablet, pill, capsule, chewing gum, wafer or the like form. In some instances, such a solid composition will typically contain one or more inert diluents or edible carriers. In certain embodiments, one or more of the following may be additionally present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

In some aspects, when the pharmaceutical composition is in the form of a capsule, for example, a gelatin capsule, it may contain, in addition to materials of the above type, a liquid carrier such as polyethylene glycol or oil.

In other aspects, the pharmaceutical composition is in the form of a liquid, for example, an elixir, syrup, solution, emulsion or suspension. In certain embodiments, the liquid may be for oral administration or for delivery by injection. In certain embodiments, when intended for oral administration, preferred compositions contain, in addition to a translational inhibitor of the invention, one or more of a sweetening agent, preservatives, dye/colorant and flavor enhancer. In certain aspects, in a composition intended to be administered by injection, one or more of a surfactant, preservative, wetting agent, dispersing agent, suspending agent, buffer, stabilizer and isotonic agent may be included.

In certain cases, the liquid pharmaceutical compositions of a translational inhibitor of the invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. In some cases, the parenteral preparation can be enclosed in ampoules, disposable syringes or multiple dose vials made of glass or plastic. In some aspects, physiological saline is a preferred adjuvant. In some embodiments, an injectable pharmaceutical composition is preferably sterile.

A liquid pharmaceutical composition of the invention intended for either parenteral or oral administration should contain an amount of a translational inhibitor of the invention such that a suitable dosage will be obtained.

In other embodiments, a pharmaceutical composition of a translational inhibitor of the invention may be intended for topical administration, in which case the carrier may suitably comprise a solution, emulsion, ointment or gel base. In certain aspects, the base, for example, may comprise one or more of the following: petrolatum, lanolin, polyethylene glycols, bee wax, mineral oil, diluents such as water and alcohol, and emulsifiers and stabilizers. In other aspects, thickening agents may be present in a pharmaceutical composition for topical administration. In certain embodiments, if intended for transdermal administration, a composition of a translational inhibitor of the invention may be included with a transdermal patch or iontophoresis device.

In yet other embodiments, the pharmaceutical composition of a translational inhibitor of the invention is intended for rectal administration, in the form, for example, of a suppository, which will melt in the rectum and release the translational inhibitor. In certain instances, a composition for rectal administration contains an oleaginous base as a suitable nonirritating excipient. Such bases include, without limitation, lanolin, cocoa butter or polyethylene glycol.

In other aspects, the pharmaceutical composition of a translational inhibitor of the invention includes various materials that modify the physical form of a solid or liquid dosage unit. For example, in certain aspects, the composition includes materials that form a coating shell around the active ingredients. In some instances, the materials that form the coating shell are typically inert, and may be selected from, for example, sugar, shellac, and other enteric coating agents. In other instances, the active ingredients are encased in a gelatin capsule.

In yet other aspects, the pharmaceutical composition of this invention in solid or liquid form include an agent that binds to a translational inhibitor of the invention and thereby assist in the delivery of the translational inhibitor. In certain cases, suitable agents that act in this capacity include a protein or a liposome.

In other aspects, a pharmaceutical composition of a translational inhibitor of the invention consist of dosage units that can be administered as an aerosol. The term aerosol is used to denote a variety of systems ranging from those of colloidal nature to systems consisting of pressurized packages. In certain embodiments, delivery is accomplished by a liquefied or compressed gas or by a suitable pump system that dispenses the active ingredients. In some embodiments, aerosols of the translational inhibitors of the invention may be delivered in single phase, bi-phasic, or tri-phasic systems in order to deliver the active ingredient(s). In other embodiments, delivery of the aerosol includes the necessary container, activators, valves, subcontainers, and the like, which together may form a kit. One skilled in the art, without undue experimentation, may determine preferred aerosol formulations and delivery modes.

A pharmaceutical composition of this invention may be prepared by methodology well-known in the pharmaceutical art. For example, a pharmaceutical composition intended to be administered by injection can be prepared by combining a translational inhibitor of the invention with a sterile solvent so as to form a solution. In certain embodiments, a surfactant may be added to facilitate the formation of a homogeneous solution or suspension. Surfactants are molecules that non-covalently interact with a compound of this invention so as to facilitate dissolution or homogeneous suspension of the compound in an aqueous delivery system.

In certain embodiments, the pharmaceutical compositions of the invention comprise one or more additional therapeutically active substances. In other embodiments, a therapeutically effective dose of the pharmaceutical compositions of the invention is administered to a subject in need thereof in combination with one or more additional therapeutically active substances. As used herein, a “combination” refers to a combination comprising a translational inhibitor of the invention and one or more additional therapeutically active substances, each of which may be administered serially (sequentially), concurrently or simultaneously.

For the purposes of the present invention, the phrase “active ingredient” refers to a translational inhibitor of the invention, or a stereoisomer, tautomer, or a pharmaceutically salt thereof. When referring to a combination, a therapeutically effective dose refers to combined amounts of the active ingredients that result in the therapeutic effect, whether administered serially or simultaneously (in the same formulation or concurrently in separate formulations). The most effective doses may generally be determined using experimental models and/or clinical trials. Design and execution of pre-clinical and clinical studies for a therapeutic agent (including when administered for prophylactic benefit) described herein are well within the skill of a person skilled in the relevant art.

In certain aspects, relative amounts of the active ingredient (i.e., a translational inhibitor of the invention), the pharmaceutically acceptable excipient, and/or any additional ingredients in a pharmaceutical composition in accordance with the present invention will vary, depending upon the identity, size, and/or condition of the subject treated and further depending upon the route by which the composition is to be administered. By way of example, the pharmaceutical composition may comprise between 0.1% and 100% (w/w), e.g., between 0.1% and 99%, between 0.5 and 50%, between 1-30%, between 5-80%, or at least about 80% (w/w) of the active ingredient.

In some embodiments, the translational inhibitors of the invention are formulated using one or more excipients, for example, to: (1) increase stability; (2) increase cell transfection; (3) permit the sustained or delayed release; (4) alter the biodistribution (e.g., target to specific tissues or cell types); (5) decrease the translation of encoded protein in vivo; and/or (6) alter the release profile of encoded protein in vivo. In certain aspects, in addition to traditional excipients such as any and all solvents, dispersion media, diluents, or other liquid vehicles, dispersion or suspension aids, surface active agents, isotonic agents, thickening or emulsifying agents, preservatives, excipients of the present invention can include, without limitation, lipidoids, liposomes, lipid nanoparticles, polymers, lipoplexes, core-shell nanoparticles, peptides, proteins, cells transfected with multimeric structures, hyaluronidase, nanoparticle mimics and combinations thereof.

In certain embodiments, the translational inhibitors of the invention are formulated using one or more liposomes, lipoplexes, or lipid nanoparticles. In some embodiments, pharmaceutical compositions of the invention include lipid nanoparticles (LNPs). In some embodiments, lipid nanoparticles are MC3-based lipid nanoparticles.

In some aspects, the number of translational inhibitors of the invention encapsulated by a lipid nanoparticle ranges from about 1 inhibitor molecule to about 100 inhibitor molecules. In other embodiments, the number of inhibitor molecules of the invention encapsulated by a lipid nanoparticle ranges from about 50 to about 500 inhibitor molecules. In other aspects, the number of inhibitor molecules of the invention encapsulated by a lipid nanoparticle ranges from about 250 to about 1000 inhibitor molecules. In yet other embodiments, the number of inhibitor molecules of the invention encapsulated by a lipid nanoparticle is greater than 1000 inhibitor molecules.

In certain embodiments, the translational inhibitors of the invention are formulated in a lipid-poly cation complex. The formation of the lipid-poly cation complex may be accomplished by methods known in the art. As a non-limiting example, the poly cation may include a cationic peptide or a polypeptide such as, but not limited to, polylysine, polyomithine and/or polyarginine. In other embodiments, the translational inhibitors of the invention are formulated in a lipid-poly cation complex which further includes a non-cationic lipid such as, but not limited to, cholesterol or dioleoylphosphatidylethanolamine (DOPE).

In certain embodiments, the translational inhibitors of the invention are formulated in a nanoparticle. In some embodiments, the nanoparticle comprises at least one lipid. In some cases, the lipid is selected from, but is not limited to, DLin-DMA, DLin-K-DMA, 98N12-5, C₁₂-200, DLin-MC 3-DMA, DLin-KC2-DMA, DODMA, PLGA, PEG, PEG-DMG, PEGylated lipids and amino alcohol lipids. In other aspects, the lipid is a cationic lipid such as, but not limited to, DLin-DMA, DLin-D-DMA, DLin-MC3-DMA, DLin-KC2-DMA, DODMA and amino alcohol lipids. In some embodiments, nanoparticle compositions also includes one or more permeability enhancer molecules, carbohydrates, polymers, surface altering agents, or other components known in the art. In other embodiments, nanoparticle compositions includes any substance useful in pharmaceutical compositions. In other aspects, nanoparticle compositions includes a lipid component and one or more additional components, such as an additional therapeutic agent.

In certain aspects, the amount of a therapeutic agent in a nanoparticle composition may depend on the size, composition, desired target and/or application, or other properties of the nanoparticle composition as well as on the properties of the therapeutic agent. For example, the amount of a translational inhibitor of the invention useful in a nanoparticle composition may depend on the size, sequence, and other characteristics of the inhibitor molecule. In certain cases, the relative amounts of a therapeutic agent and other elements (e.g., lipids) in a nanoparticle composition may also vary. In some embodiments, the wt/wt ratio of the lipid component to a therapeutic agent in a nanoparticle composition may be from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. In certain aspects, the wt/wt ratio of the lipid component to a therapeutic agent may be from about 10:1 to about 40:1. In specific aspects, the wt/wt ratio is about 20:1.

### Therapeutic Uses

The translational inhibitors of the invention, or their pharmaceutically acceptable salts, are administered in a therapeutically effective amount, which will vary depending upon a variety of factors including the activity of the specific compound employed; the metabolic stability and length of action of the translational inhibitor; the age, body weight, general health, sex, and diet of the patient; the mode and time of administration; the rate of excretion; the drug combination; the severity of the particular disorder or condition; and the subject undergoing therapy.

The translational inhibitors of the invention, or pharmaceutically acceptable salt thereof may also be administered simultaneously with, prior to, or after administration of one or more other therapeutic agents. Such combination therapy includes administration of a single pharmaceutical dosage formulation which contains a translational inhibitor of the invention and one or more additional active agents, as well as administration of a translational inhibitor of the invention and each active agent in its own separate pharmaceutical dosage formulation. For example, a translational inhibitor of the invention and the other active agent can be administered to the patient together in a single oral dosage composition such as a tablet or capsule, or each agent administered in separate oral dosage formulations. Where separate dosage formulations are used, the translational inhibitors of the invention and one or more additional active agents can be administered at essentially the same time, i.e., concurrently, or at separately staggered times, i.e., sequentially; combination therapy is understood to include all these regimens.

In certain embodiments, the present invention also contemplates the production of the translational inhibitors of the invention for use as therapeutic agents in a pharmaceutical composition, or the introduction of the translational inhibitors of the invention into cells of a subject to treat a medical condition.

In certain aspects, a cell population is contacted with an effective amount of a composition containing a translational inhibitor of the invention. In certain embodiments, an effective amount of the composition of a translational inhibitor of the invention is provided based, at least in part, on the target tissue, target cell type, means of administration, and other determinants. In certain aspects, the subject to whom a translational inhibitor of the invention is administered suffers from or is at risk of developing a disease, disorder, or deleterious condition. In some embodiments, methods of identifying, diagnosing, and classifying subjects on these bases include clinical diagnosis, biomarker levels, genome-wide association studies (GWAS), and other methods known in the art.

In certain embodiments, the translational inhibitors are useful for inhibiting the activity of eIF4E and/or can be useful in analyzing eIF4E signaling activity in model systems and/or for preventing, treating, or ameliorating a symptom associated with a disease, disorder, or pathological condition involving eIF4E. A translational inhibitor which inhibits the activity of eIF4E will be useful in preventing, treating, ameliorating, or reducing the symptoms or progression of diseases of uncontrolled cell growth, proliferation and/or survival, inappropriate cellular immune responses, or inappropriate cellular inflammatory responses or diseases which are accompanied with uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses is mediated by eIF4E.

Thus, in certain embodiments, the present invention provides a method for treating a eIF4E-dependent condition in a subject in need thereof comprising administering to the subject (i) a therapeutically effective amount of the translational inhibitor of the invention, or a stereoisomer, tautomer or pharmaceutically acceptable salt thereof, or (ii) a pharmaceutical composition of the invention. In certain instances, an effective amount of a composition containing a translational inhibitor of the invention is administered to the subject using the delivery methods described herein. In some embodiments, the cell in which a translational inhibitor of the invention is localized, or the tissue in which the cell is present, is targeted with one or more than one rounds of administration of the translational inhibitor of the invention. In certain aspects, the subject is a mammal. In other aspects, the subject is a human. In certain embodiments, the translational inhibitors of the invention is capable of treating, preventing, or ameliorating an eIF4E-dependent condition when administered to a subject in need thereof.

In some embodiments, the eIF4E-dependent condition is a disease including, but not limited to, hyperproliferative disease, autoimmune disease, diabetes, neurodegenerative disease, inflammatory disease, viral infection, cardiovascular disease, metabolic disease, genetic diseases (including, but not limited to, Alzheimer's, Parkinson's, Fragile X Syndrome and autism), or any combination thereof.

In certain aspects, the disease is a hyperproliferative disease. In specific aspects, the hyperproliferative disease is cancer. In specific embodiments, the cancer includes, without limitation, solid tumor, melanoma, multiple melanoma, non-small cell lung cancer, renal cell carcinoma, renal cancer, a hematological cancer, prostate cancer, castration-resistant prostate cancer, colon cancer, rectal cancer, gastric cancer, esophageal cancer, bladder cancer, head and neck cancer, thyroid cancer, breast cancer, triple-negative breast cancer, ovarian cancer, cervical cancer, lung cancer, urothelial cancer, pancreatic cancer, glioblastoma, hepatocellular cancer, myeloma, multiple myeloma, leukemia, B-cell lymphoma, T-cell lymphoma, hairy cell lymphoma, Hodgkin's lymphoma, non-Hodgkin's lymphoma, Burkitt's lymphoma, myelodysplastic syndrome, brain cancer, CNS cancer, malignant glioma, or any combination thereof.

In certain embodiments, the eIF4E-dependent condition is a condition including, but not limited to solid tumor, melanoma, multiple melanoma, non-small cell lung cancer, renal cell carcinoma, renal cancer, a hematological cancer, prostate cancer, castration-resistant prostate cancer, colon cancer, rectal cancer, gastric cancer, esophageal cancer, bladder cancer, head and neck cancer, thyroid cancer, breast cancer, triple-negative breast cancer, ovarian cancer, cervical cancer, lung cancer, urothelial cancer, pancreatic cancer, glioblastoma, hepatocellular cancer, myeloma, multiple myeloma, leukemia, B-cell lymphoma, T-cell lymphoma, hairy cell lymphoma, Hodgkin's lymphoma, non-Hodgkin's lymphoma, Burkitt's lymphoma, myelodysplastic syndrome, brain cancer, CNS cancer, malignant glioma, Alzheimer's, Parkinson's, Fragile X Syndrome, autism disorders, and any combination thereof.

In general, the types of cancer that may be treated using the translational inhibitors of the invention include, but are not limited to: adenocarcinoma of the breast, prostate, and colon; all forms of bronchogenic carcinoma of the lung; myeloid; melanoma; hepatoma; neuroblastoma; papilloma; apudoma; choristoma; branchioma; malignant carcinoid syndrome; carcinoid heart disease; and carcinoma (e.g., Walker, basal cell, basosquamous, Brown-Pearce, ductal, Ehrlich tumor, Krebs 2, merkel cell, mucinous, non-small cell lung, oat cell, papillary, scirrhous, bronchiolar, bronchogenic, squamous cell, and transitional cell). Additional types of cancers that may be treated include: histiocytic disorders; leukemia; histiocytosis malignant; Hodgkin's disease; immunoproliferative small; non-Hodgkin's lymphoma; T-cell lymphoma, B-cell lymphoma, hairy cell lymphoma, Burkitt's lymphoma, plasmacytoma; reticuloendotheliosis; melanoma; chondroblastoma; chondroma; chondrosarcoma; fibroma; fibrosarcoma; giant cell tumors; histiocytoma; lipoma; liposarcoma; mesothelioma; myxoma; myxosarcoma; osteoma; osteosarcoma; chordoma; craniopharyngioma; dysgerminoma; hamartoma; mesenchymoma; mesonephroma; myosarcoma; ameloblastoma; cementoma; odontoma; teratoma; thymoma; trophoblastic tumor.

Other cancers that can be treated using the translational inhibitors of the invention include without limitation adenoma; cholangioma; cholesteatoma; cyclindroma; cystadenocarcinoma; cystadenoma; granulosa cell tumor; gynandroblastoma; hepatoma; hidradenoma; islet cell tumor; Leydig cell tumor; papilloma; sertoli cell tumor; theca cell tumor; leimyoma; leiomyosarcoma; myoblastoma; myomma; myosarcoma; rhabdomyoma; rhabdomyosarcoma; ependymoma; ganglioneuroma; glioma; medulloblastoma; meningioma; neurilemmoma; neuroblastoma; neuroepithelioma; neurofibroma; neuroma; paraganglioma; paraganglioma nonchromaffin.

In some embodiments the translational inhibitors of the invention are candidate therapeutic agents for the treatment of cancers such as angiokeratoma; angiolymphoid hyperplasia with eosinophilia; angioma sclerosing; angiomatosis; glomangioma; hemangioendothelioma; hemangioma; hemangiopericytoma; hemangiosarcoma; lymphangioma; lymphangiomyoma; lymphangiosarcoma; pinealoma; carcinosarcoma; chondrosarcoma; cystosarcoma phyllodes; fibrosarcoma; hemangiosarcoma; leiomyosarcoma; leukosarcoma; liposarcoma; lymphangiosarcoma; myosarcoma; myxosarcoma; ovarian carcinoma; rhabdomyosarcoma; sarcoma; neoplasms; nerofibromatosis; and cervical dysplasia.

Furthermore, the inventive translational inhibitors and their pharmaceutical compositions are candidate therapeutics for the prophylaxis and/or therapy of cytokine related diseases, such as inflammatory diseases, allergies, or other conditions associated with proinflammatory cytokines. Exemplary inflammatory diseases include without limitation, chronic or acute inflammation, inflammation of the joints such as chronic inflammatory arthritis, rheumatoid arthritis, psoriatic arthritis, osteoarthritis, juvenile rheumatoid arthritis, Reiter's syndrome, rheumatoid traumatic arthritis, rubella arthritis, acute synovitis and gouty arthritis; inflammatory skin diseases such as sunburn, psoriasis, erythrodermic psoriasis, pustular psoriasis, eczema, dermatitis, acute or chronic graft formation, atopic dermatitis, contact dermatitis, urticaria and scleroderma; inflammation of the gastrointestinal tract such as inflammatory bowel disease, Crohn's disease and related conditions, ulcerative colitis, colitis, and diverticulitis; nephritis, urethritis, salpingitis, oophoritis, endomyometritis, spondylitis, systemic lupus erythematosus and related disorders, multiple sclerosis, asthma, meningitis, myelitis, encephalomyelitis, encephalitis, phlebitis, thrombophlebitis, respiratory diseases such as asthma, bronchitis, chronic obstructive pulmonary disease (COPD), inflammatory lung disease and adult respiratory distress syndrome, and allergic rhinitis; endocarditis, osteomyelitis, rheumatic fever, rheumatic pericarditis, rheumatic endocarditis, rheumatic myocarditis, rheumatic mitral valve disease, rheumatic aortic valve disease, prostatitis, prostatocystitis, spondoarthropathies ankylosing spondylitis, synovitis, tenosynovotis, myositis, pharyngitis, polymyalgia rheumatica, shoulder tendonitis or bursitis, gout, pseudo gout, vasculitides, inflammatory diseases of the thyroid selected from granulomatous thyroiditis, lymphocytic thyroiditis, invasive fibrous thyroiditis, acute thyroiditis; Hashimoto's thyroiditis, Kawasaki's disease, Raynaud's phenomenon, Sjogren's syndrome, neuroinflammatory disease, sepsis, conjunctivitis, keratitis, iridocyclitis, optic neuritis, otitis, lymphoadenitis, nasopaharingitis, sinusitis, pharyngitis, tonsillitis, laryngitis, epiglottitis, bronchitis, pneumonitis, stomatitis, gingivitis. oesophagitis, gastritis, peritonitis, hepatitis, cholelithiasis, cholecystitis, glomerulonephritis, goodpasture's disease, crescentic glomerulonephritis, pancreatitis, endomyometritis, myometritis, metritis, cervicitis, endocervicitis, exocervicitis, parametritis, tuberculosis, vaginitis, vulvitis, silicosis, sarcoidosis, pneumoconiosis, pyresis, inflammatory polyarthropathies, psoriatric arthropathies, intestinal fibrosis, bronchiectasis and enteropathic arthropathies.

Yet further, the inventive translational inhibitors and their pharmaceutical compositions are candidate therapeutics for the prophylaxis and/or therapy of fibrotic diseases, such as various forms of fibrosis, fibromas or any disease giving rise to fibrosis whether as a main or a secondary symptom. Exemplary fibrotic diseases include without limitation, viral hepatitis, hepatic fibrosis, schistosomiasis, steatohepatitis (alcoholic or non-alcoholic), cirrhosis, idiopathic pulmonary fibrosis (IPF), systemic sclerosis (scleroderma), nephrogenic systemic fibrosis (NSF), diabetes, untreated hypertension, heart attack, hypertension, atherosclerosis, restenosis, macular degeneration, retinal and vitreal retinopathy, keloids, hypertrophic scars, Crohn's disease and Alzheimer's disease.

Although inflammation is the unifying pathogenic process of these diseases, current therapies only treat the symptoms of the disease and not the underlying cause of inflammation. The compositions of the present invention are useful for the treatment and/or prophylaxis of inflammatory diseases and related complications and disorders.

### Combination Therapies

In certain embodiments the translational inhibitors of the invention are administered to a subject suffering from an eIF4E-dependent disorder in conjunction with other conventional therapies such as radiation treatment or surgery. Radiation therapy is well-known in the art and includes X-ray therapies, such as gamma-irradiation, and radiopharmaceutical therapies.

In certain embodiments the translational inhibitors of the invention are used with at least one anti-cancer agent. Anti-cancer agents include chemotherapeutic drugs. A chemotherapeutic agent includes, but is not limited to, an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), and a DNA repair inhibitor.

Illustrative chemotherapeutic agents include, without limitation, the following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, Cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, temozolamide, teniposide, triethylenethiophosphoramide and etoposide (VP 16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab, rituximab); chimeric antigen receptors; cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers, toxins such as Cholera toxin, ricin, Pseudomonas exotoxin, Bordetella pertussis adenylate cyclase toxin, or diphtheria toxin, and caspase activators; and chromatin disruptors.

Where the subject is suffering from or at risk of suffering from an autoimmune disease, an inflammatory disease, or an allergy disease, a translational inhibitor of the invention can be used in with one or more of the following therapeutic agents in any combination: immunosuppressants (e.g., tacrolimus, cyclosporin, rapamicin, methotrexate, cyclophosphamide, azathioprine, mercaptopurine, mycophenolate, or FTY720), glucocorticoids (e.g., prednisone, cortisone acetate, prednisolone, methylprednisolone, dexamethasone, betamethasone, triamcinolone, beclometasone, fludrocortisone acetate, deoxycorticosterone acetate, aldosterone), non-steroidal anti-inflammatory drugs (e.g., salicylates, arylalkanoic acids, 2-arylpropionic acids, N-arylanthranilic acids, oxicams, coxibs, or sulphonanilides), Cox-2-specific inhibitors (e.g., valdecoxib, celecoxib, or rofecoxib), leflunomide, gold thioglucose, gold thiomalate, aurofin, sulfasalazine, hydroxychloroquinine, minocycline, TNF-α binding proteins (e.g., infliximab, etanercept, or adalimumab), abatacept, anakinra, interferon-β, interferon-γ, interleukin-2, allergy vaccines, antihistamines, antileukotrienes, beta-agonists, theophylline, or anticholinergics.

Where the subject is suffering from or at risk of suffering from a thromboembolic disorder (e.g., stroke), the subject can be treated with a translational inhibitor of the invention in any combination with one or more other anti-thromboembolic agents. Examples of anti-thromboembolic agents include, but are not limited any of the following: thrombolytic agents (e.g., alteplase anistreplase, streptokinase, urokinase, or tissue plasminogen activator), heparin, tinzaparin, warfarin, dabigatran (e.g., dabigatran etexilate), factor Xa inhibitors (e.g., fondaparinux, draparinux, rivaroxaban, DX-9065a, otamixaban, LY517717, or YM150), ticlopidine, clopidogrel, CS-747 (prasugrel, LY640315), ximelagatran, or BIBR 1048.

Examples of natural products useful in combination with a translational inhibitor of the invention include but are not limited to vinca alkaloids (e.g., vinblastin, vincristine), epipodophyllotoxins (e.g., etoposide), antibiotics (e.g., daunorubicin, doxorubicin, bleomycin), enzymes (e.g., L-asparaginase), or biological response modifiers (e.g., interferon alpha).

Examples of alkylating agents that can be employed in combination a translational inhibitor of the invention include, but are not limited to, nitrogen mustards (e.g., mechloroethamine, cyclophosphamide, chlorambucil, melphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.). Examples of antimetabolites include, but are not limited to folic acid analog (e.g., methotrexate), or pyrimidine analogs (e.g., fluorouracil, floxuridine, Cytarabine), purine analogs (e.g., mercaptopurine, thioguanine, pentostatin.

Examples of hormones and antagonists useful in combination with a translational inhibitor of the invention include, but are not limited to, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide). Other agents that can be used in the methods and compositions described herein for the treatment or prevention of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide).

In certain embodiments a translational inhibitor of the invention is used simultaneously, in the same formulation or in separate formulations, or sequentially with an additional agent(s) as part of a combination therapy regimen.

In certain embodiments, the translational inhibitors of the invention their corresponding salts and pharmaceutically acceptable compositions are candidate therapeutics for treating brain related disorders which include, without limitation, autism, Fragile X-syndrome, Parkinson's disease and Alzheimer's disease.

The invention also supports the use of the translational inhibitors of the invention or a pharmaceutically acceptable formulation of the translational inhibitors as an inhibitor of eIF4E activity. Such inhibition is achieved by contacting a cell expressing eIF4E with a translational inhibitor of the invention or a pharmaceutically acceptable formulation, to lower or inhibit eIF4E activity, to provide therapeutic efficacy for a eIF4E-dependent condition in a subject in need thereof.

Other aspects of the present invention relate to transplantation of cells comprising the translational inhibitors of the invention to a subject. Administration of cells to subjects is known to those of ordinary skill in the art, such as local implantation (e.g., topical or subcutaneous administration), organ delivery or systemic injection (e.g., intravenous injection or inhalation), as is the formulation of cells in pharmaceutically acceptable carrier. In certain embodiments, compositions comprising the translational inhibitors of the invention are formulated for administration intramuscularly, transarterially, intraperitoneally, intravenously, intranasally, subcutaneously, endoscopically, transdermally, or intrathecally. In some embodiments, the compositions are formulated for extended release.

In other embodiments, the invention provides a method for attenuating or inhibiting the activity of eIF4E in at least one cell overexpressing eIF4E, comprising contacting the at least one cell with the translational inhibitor of the invention, or a stereoisomer, tautomer or pharmaceutically acceptable salt thereof.

In additional embodiments, the invention provides a method for inhibiting translation in at least one cell overexpressing eIF4E, comprising contacting the at least one cell with the translational inhibitor of the invention, or a stereoisomer, tautomer or pharmaceutically acceptable salt thereof.

In certain aspects, the at least one cell includes, without limitation, a colon cancer cell, a gastric cancer cell, a thyroid cancer cell, a lung cancer cell, a leukemia cell, a B-cell lymphoma, a T-cell lymphoma, a hairy cell lymphoma, Hodgkin's lymphoma cell, non-Hodgkin's lymphoma cell, Burkitt's lymphoma cell, a pancreatic cancer cell, a melanoma cell, a multiple melanoma cell, a brain cancer cell, a CNS cancer cell, a renal cancer cell, a prostate cancer cell, an ovarian cancer cell, or a breast cancer cell. In certain embodiments, the target cells are epithelial cells, such as the lung, and methods of administration are determined in view of the target tissue; i.e., for lung delivery, the translational inhibitors of the invention are formulated for administration by inhalation.

In some aspects are provided a method of treating, preventing, or ameliorating a disease in a subject comprising administering to the subject a therapeutically effective amount of a translational inhibitor of the invention. In certain aspects, the disease includes, but is not limited to, hyperproliferative disease, inflammatory disease, viral infection, cardiovascular disease, genetic disease, and autoimmune disease.

### Dosage

Generally, a therapeutic agent is administered at a therapeutically effective amount or dose. It will be appreciated, however, that a therapeutically effective amount or dose will vary according to several factors, including the chosen route of administration, formulation of the composition, patient response, severity of the condition, the subject's age, sex, body weight, general health condition, diet, individual response of the subject to be treated, time of administration, severity of the disease to be treated, the activity of particular compound applied, dosage form, mode of application and concomitant medication, and the judgment of the prescribing physician. The therapeutically effective amount for a given situation will readily be determined by routine experimentation and is within the skills and judgment of the ordinary clinician or physician. In any case, the translational inhibitor of the invention or composition will be administered at dosages and in a manner which allows a therapeutically effective amount to be delivered based upon a subject's unique condition.

The dosage can be increased or decreased over time, as required by an individual subject. In certain instances, a patient initially is given a low dose, which is then increased to an efficacious dosage tolerable to the subject. In addition, a patient may be given a plurality of doses over a determined period of time and in particular time increments (such as daily, weekly, biweekly, monthly, quarterly, biannually, annually or the like). Determination of an effective amount or dosing regimen is well within the capability of those skilled in the art.

Therapeutically effective dosages of a translational inhibitor of the invention will generally range from about 1 to 2000 mg/day, from about 10 to about 1000 mg/day, from about 10 to about 500 mg/day, from about 10 to about 250 mg/day, from about 10 to about 100 mg/day, or from about 10 to about 50 mg/day. The therapeutically effective dosages may be administered in one or multiple doses.

In certain embodiments, a translational inhibitor in accordance with the invention is administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, or prophylactic effect. In certain embodiments, the desired dosage is delivered three times a day, two times a day, once a day, every other day, every third day, every week, every two weeks, every three weeks, or every four weeks. In some embodiments, the desired dosage is delivered using multiple administrations (e.g., two, three, four, five, six, seven, eight, nine, ten, eleven, twelve, thirteen, fourteen, or more administrations).

The route of administration of a translational inhibitor or pharmaceutical composition of the invention can be oral, intraperitoneal, transdermal, subcutaneous, by intravenous or intramuscular injection, by inhalation, topical, intralesional, infusion; liposome-mediated delivery; topical, intrathecal, gingival pocket, rectal, intrabronchial, nasal, transmucosal, intestinal, ocular or otic delivery, or any other methods known in the art.

### Assays

In certain embodiments, the translational inhibitors of the invention, once produced, can be characterized using a variety of assays known to those skilled in the art to determine whether they have biological activity. For example, the translational inhibitors of the invention can be characterized by conventional assays, including but not limited to protein production assays (e.g., cell-free translation assays or cell based expression assays), degradation assays, cell culture assays (e.g., of neoplastic cells), animal models (e.g., rats, mice, rabbits, dogs, or pigs) and the like, to determine whether they have a predicted activity, e.g., eIF4E-binding activity and/or translation inhibiting activity.

In some embodiments, high-throughput screening can be used to speed up analysis using such assays. As a result, it can be possible to rapidly screen the molecules described herein for activity, using high-throughput screening. General methodologies for performing high-throughput screening are well known in the art.

### Kits

In certain aspects, kits including, a translational inhibitor of the invention are also contemplated. In certain embodiments, the kit further comprises a buffer.

Various aspects of the present invention are illustrated by the following non-limiting examples. The examples are for illustrative purposes and are not a limitation on any practice of the present invention. It will be understood that variations and modifications can be made without departing from the spirit and scope of the invention. One of ordinary skill in the art readily knows how to synthesize or commercially obtain the reagents and components described herein.

### EXAMPLES

### Example 1

### 4-fluorophenyl 2-(7-(5-chloro-2-(2-(5-cyano-6-((1-(3,3-difluorocyclobutyl)piperidin-4-yl)(methyl)amino)-2-methyl-4-oxopyrido[3,4-d]pyrimidin-3(4H)-yl)ethoxy)phenyl)thieno[3,2-b]pyridin-3-yl)acetate (Example 1)

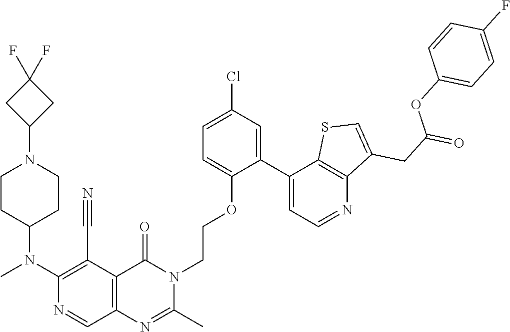 [SMILES: Cc1nc2cnc(N(C)C3CCN(C4CC(F)(F)C4)CC3)c(C#N)c2c(=O)n1CCOc1ccc(Cl)cc1-c1ccnc2c(CC(=O)Oc3ccc(F)cc3)csc12]

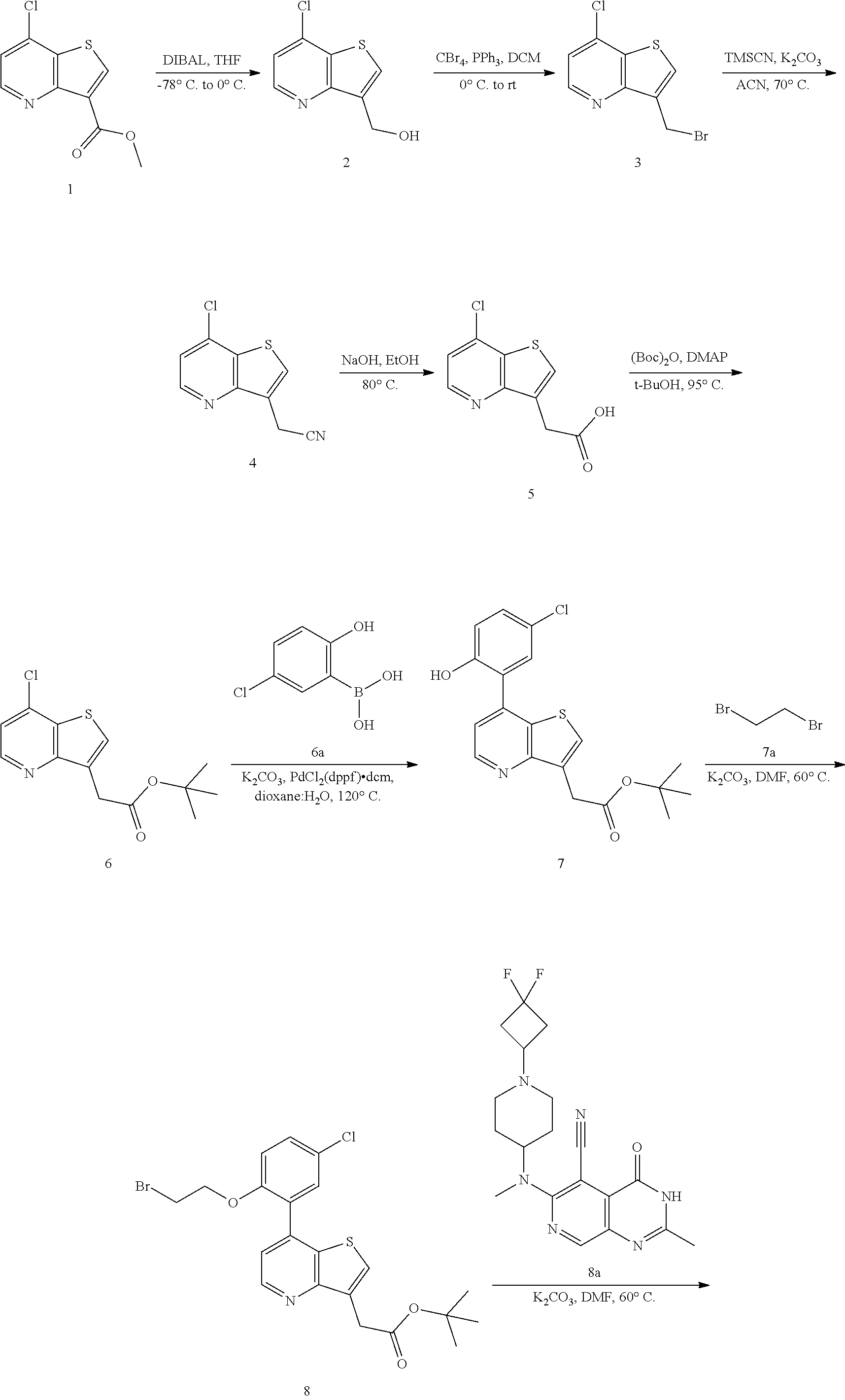

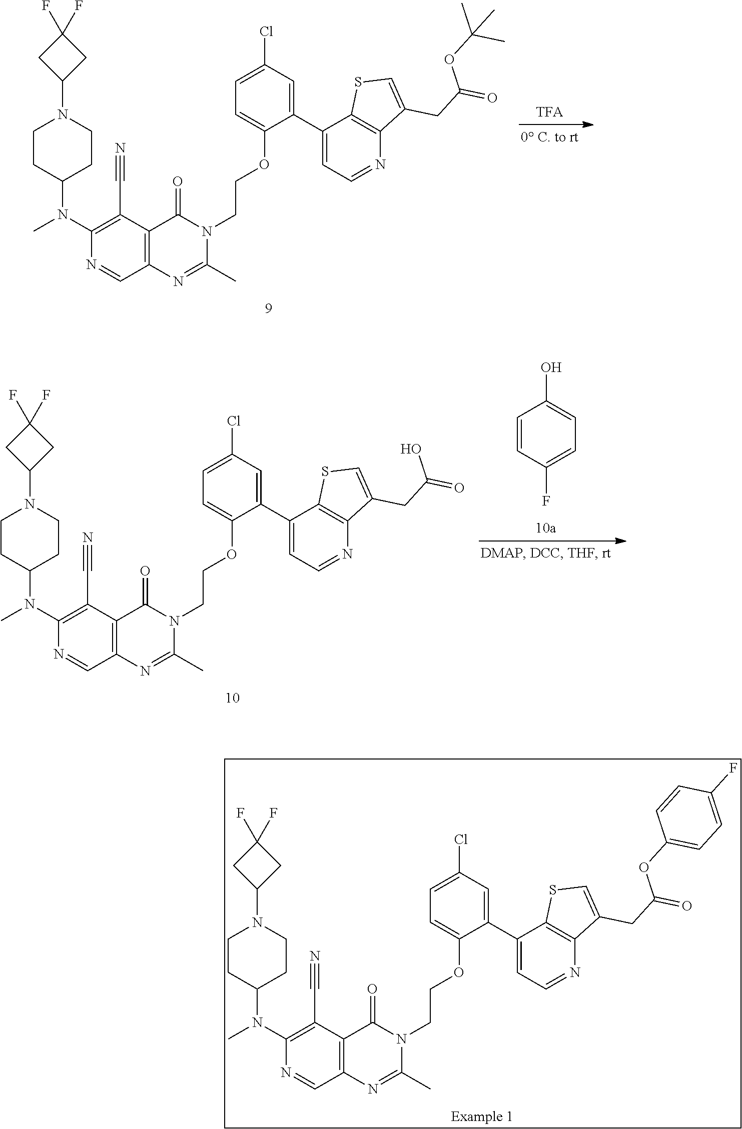 [SMILES: Cc1nc2cnc(N(C)C3CCN(C4CC(F)(F)C4)CC3)c(C#N)c2c(=O)n1CCOc1ccc(Cl)cc1-c1ccnc2c(CC(=O)OC(C)(C)C)csc12 ; Cc1nc2cnc(N(C)C3CCN(C4CC(F)(F)C4)CC3)c(C#N)c2c(=O)n1CCOc1ccc(Cl)cc1-c1ccnc2c(CC(=O)O)csc12 ; Oc1ccc(F)cc1 ; Cc1nc2cnc(N(C)C3CCN(C4CC(F)(F)C4)CC3)c(C#N)c2c(=O)n1CCOc1ccc(Cl)cc1-c1ccnc2c(CC(=O)Oc3ccc(F)cc3)csc12]

### Synthesis of (7-chlorothieno[3,2-b]pyridin-3 yl)methanol (2)

A solution of methyl 7-chlorothieno[3,2-b]pyridine-3-carboxylate (1, 10.0 g, 44.24 mmol) in tetrahydrofuran (100 ml) was cooled at −78° C., diisobutylalluminium hydride (1.0 M in toluene, 132.7 ml, 132.7 mmol) was added and reaction mixture was stirred at −78° C. for 1 h. Then, the mixture was allowed to warm to 0° C. over an hour. After completion, the reaction mixture was quenched with saturated ammonium chloride solution and extracted with ethyl acetate. The organic layer was dried over anhydrous sodium sulphate, filtered and concentrated. The crude product was purified by column chromatography using silica gel (100-200 mesh) and 0-10% ethyl acetate in hexane to afford (7-chlorothieno[3,2-b]pyridin-3-yl)methanol (2) as a light green solid. Yield: 5.50 g, MS (ESI) m/z 199.98 [M+1]⁺.

### Synthesis of 3-(bromomethyl)-7-chlorothieno[3,2-b]pyridine (3)

To a solution of (7-chlorothieno[3,2-b]pyridin-3-yl)methanol (2, 5.50 g, 20.99 mmol) in dichloromethane (100 mL), triphenylphosphine (8.28 g, 31.48 mmol) was added at 0° C. and then stirred for 15 min. & then tetabromomethane (10.42 g, 31.48 mmol) was added and mixture was stirred at rt for 2 h. After completion, water was added to reaction mixture and extracted with dichloromethane. The organic layer was dried over anhydrous sodium sulphate, filtered and concentrated. The crude product was purified by column chromatography using silica gel (100-200 mesh) and 10-20% ethyl acetate in hexane to afford 3-(bromomethyl)-7-chlorothieno[3,2-b]pyridine (3) as a white solid. Yield: 5.0 g, 76%; MS (ESI) m/z 264.00 [M+1]⁺.

### Synthesis of 2-(7-chlorothieno[3,2-b]pyridin-3-yl)acetonitrile (4)

To a solution of 3-(bromomethyl)-7-chlorothieno[3,2-b]pyridine (3, 5.0 g, 24.03 mmol) in acetonitrile (50 mL), trimethylsilyl cyanide (4.78 ml, 38.16 mmol) and potassium carbonate (7.90 g, 57.25 mmol) were added and the mixture was heated at 70° C. for 16 h. After completion, water was added to reaction mixture and extracted with ethyl acetate. The organic layer was dried over anhydrous sodium sulphate, filtered and concentrated to get crude which was purified by column chromatography using silica gel (100-200 mesh) and 10-20% ethyl acetate in hexane to afford 2-(7-chlorothieno[3,2-b]pyridin-3-yl)acetonitrile (4) as a white solid. Yield: 3.70 g, 94%; MS (ESI) m/z 208.96 [M+1]⁺.

### Synthesis of 2-(7-chlorothieno[3,2-b]pyridin-3-yl)acetic acid (5)

To a solution of 2-(7-chlorothieno[3,2-b]pyridin-3-yl)acetonitrile (4), 3.70 g, 17.78 mmol) in ethanol (40 mL), 4M sodium hydroxide (22.23 ml, 88.94 mmol) was added and the mixture was heated at 80° C. for 16 h. After completion, the reaction mixture evaporated under reduced pressure and acidified with 1N hydrochloric acid, the precipitate formed was filtered through sintered funnel and dried to afford 2-(7-chlorothieno[3,2-b]pyridin-3-yl)acetic acid (5) as a white solid. Yield: 3.0 g, 75%; MS (ESI) m/z 227.96 [M+1]⁺.

### Synthesis of tert-butyl 2-(7-chlorothieno[3,2-b]pyridin-3-yl)acetate (6)

To a solution of 2-(7-chlorothieno[3,2-b]pyridin-3-yl)acetic acid (5, 3.0 g, 13.21 mmol) in tert-butanol (30 mL), boc-anhydride (9.60 ml, 39.64 mmol) and 4-dimethylaminopyridine (1.61 g, 13.21 mmol) were added and the mixture was heated at 95° C. for 16 h. After completion, water was added to reaction mixture and extracted with ethyl acetate. The organic layer was dried over anhydrous sodium sulphate, filtered and concentrated to get crude which was purified by column chromatography using silica gel (100-200 mesh) and 0-10% ethyl acetate in hexane to afford tert-butyl 2-(7-chlorothieno[3,2-b]pyridin-3-yl)acetate (6) as a colourless sticky solid. Yield: 1.6 g, 42%; MS (ESI) m/z 284.03 [M+1]⁺.

### Synthesis of tert-butyl 2-(7-(5-chloro-2-hydroxyphenyl)thieno[3,2-b]pyridin-3-yl)acetate (7)

A solution of tert-butyl 2-(7-chlorothieno[3,2-b]pyridin-3-yl)acetate (6, 1.60 g, 5.65 mmol), (5-chloro-2-hydroxyphenyl)boronic acid (6a, 1.94 g, 11.30 mmol) and potassium carbonate (2.34 g, 16.96 mmol) in 1,4-dioxane:water (4:1, 16 mL) was degassed under nitrogen for 5 minutes. Then, [1,1′-bis(diphenylphosphino)ferrocene]dichloropalladium(II) (0.226 g, 0.282 mmol) was added and the reaction mixture was heated at 120° C. for 16 h. The reaction mixture was cooled to room temperature; concentrated and the crude product was purified by column chromatography using silica gel (100-200 mesh) and 10-20% ethyl acetate in hexane to afford tert-butyl 2-(7-(5-chloro-2-hydroxyphenyl)thieno[3,2-b]pyridin-3-yl)acetate (7) as an off white solid. Yield: 1.80 g, 84.9%; MS (ESI) m/z 376.03 [M+1]⁺.

### Synthesis of tert-butyl 2-(7-(2-(2-bromoethoxy)-5-chlorophenyl)thieno[3,2-b]pyridin-3-yl)acetate (8)

To a solution of tert-butyl 2-(7-(5-chloro-2-hydroxyphenyl)thieno[3,2-b]pyridin-3-yl)acetate (7, 1.80 g, 4.80 mmol) in N,N-dimethyl formamide (18 mL), potassium carbonate (3.31 g, 24.0 mmol) and 1,2-dibromoethane (1.24 ml, 14.4 mmol) were added and reaction mixture heated at 60° C. for 16 h. After completion, water was added to reaction mixture and extracted with ethyl acetate. The organic layer was dried over anhydrous sodium sulphate, filtered and concentrated to get crude which was purified by column chromatography using silica gel (100-200 mesh) and 0-10% ethyl acetate in hexane to afford tert-butyl 2-(7-(2-(2-bromoethoxy)-5-chlorophenyl)thieno[3,2-b]pyridin-3-yl)acetate (8) as a white solid. Yield: 1.4 g, 60%; MS (ESI) m/z 483.76 [M+1]⁺.

### Synthesis of tert-butyl 2-(7-(5-chloro-2-(2-(5-cyano-6-((1-(3,3-difluorocyclobutyl)piperidin-4-yl)(methyl)amino)-2-methyl-4-oxopyrido[3,4-d]pyrimidin-3(4H)-yl)ethoxy)phenyl)thieno[3,2-b]pyridin-3-yl)acetate (9)

To a solution of 6-((1-(3,3-difluorocyclobutyl)piperidin-4-yl)(methyl)amino)-2-methyl-4-oxo-3,4-dihydropyrido[3,4-d]pyrimidine-5-carbonitrile (8a, 1.4 g, 3.60 mmol) in N,N-dimethyl formamide (14 mL), potassium carbonate (1.49 g, 10.82 mmol) and tert-butyl 2-(7-(2-(2-bromoethoxy)-5-chlorophenyl)thieno[3,2-b]pyridin-3-yl)acetate (8, 1.73 g, 3.60 mmol) were added and the mixture heated at 60° C. for 16 h. After completion, water was added to reaction mixture and extracted with ethyl acetate. The organic layer was dried over anhydrous sodium sulphate, filtered and concentrated. The crude product was purified by column chromatography using silica gel (100-200 mesh) and 30-40% ethyl acetate in hexane to afford tert-butyl 2-(7-(5-chloro-2-(2-(5-cyano-6-((1-(3,3-difluorocyclobutyl)piperidin-4-yl)(methyl)amino)-2-methyl-4-oxopyrido[3,4-d]pyrimidin-3(4H)-yl)ethoxy)phenyl)thieno[3,2-b]pyridin-3-yl)acetate (9) as a yellow solid. Yield: 1.50 g, 52%; MS (ESI) m/z 790.52 [M+1]⁺.

### Synthesis of 2-(7-(5-chloro-2-(2-(5-cyano-6-((1-(3,3-difluorocyclobutyl)piperidin-4-yl)(methyl)amino)-2-methyl-4-oxopyrido[3,4-d]pyrimidin-3(4H)-yl)ethoxy)phenyl)thieno[3,2-b]pyridin-3-yl)acetic acid (TFA salt) (10)

A solution of tert-butyl 2-(7-(5-chloro-2-(2-(5-cyano-6-((1-(3,3-difluorocyclobutyl)piperidin-4-yl)(methyl)amino)-2-methyl-4-oxopyrido[3,4-d]pyrimidin-3(4H)-yl)ethoxy)phenyl)thieno[3,2-b]pyridin-3-yl)acetate (9, 1.50 g, 18.98 mmol) in trifluoroacetic acid (15 mL) was stirred at room temperature for 2 h. After completion, the reaction mixture was concentrated; the residue was washed with diethyl ether and dried under reduced pressure to afford 2-(7-(5-chloro-2-(2-(5-cyano-6-((1-(3,3-difluorocyclobutyl)piperidin-4-yl)(methyl)amino)-2-methyl-4-oxopyrido[3,4-d]pyrimidin-3(4H)-yl)ethoxy)phenyl)thieno[3,2-b]pyridin-3-yl)acetic acid (TFA salt) (10) as an yellow solid. Yield: 1.80 g, crude; MS (ESI) m/z 734.02 [M+1]⁺. This crude product was taken as such for next step without purification.

### Synthesis of 4-fluorophenyl 2-(7-(5-chloro-2-(2-(5-cyano-6-((1-(3,3-difluorocyclobutyl)piperidin-4-yl)(methyl)amino)-2-methyl-4-oxopyrido[3,4-d]pyrimidin-3(4H)-yl)ethoxy)phenyl)thieno[3,2-b]pyridin-3-yl)acetate. (Example 1)

A solution of 2-(7-(5-chloro-2-(2-(5-cyano-6-((1-(3,3-difluorocyclobutyl)piperidin-4-yl)(methyl)amino)-2-methyl-4-oxopyrido[3,4-d]pyrimidin-3(4H)-yl)ethoxy)phenyl)thieno[3,2-b]pyridin-3-yl)acetic acid (10, 0.20 g, 0.272 mmol) in tetrahydrofuran (4 mL) was cooled to 0° C. 4-fluorophenol (10a, 0.030 g, 0.272 mmol), N,N′-dicyclohexylcarbodiimide (0.067 g, 0.327 mmol) and 4-dimethylaminopyridine (0.099 g, 0.818 mmol) were added and the mixture was stirred at room temperature for 3 h. After completion, the reaction mixture was diluted with ethyl acetate, washed with water and 5% aqueous citric acid solution. The organic layer was dried over anhydrous sodium sulphate, filtered and concentrated. The crude product was purified by preparative HPLC to afford 4-fluorophenyl 2-(7-(5-chloro-2-(2-(5-cyano-6-((1-(3,3-difluorocyclobutyl)piperidin-4-yl)(methyl)amino)-2-methyl-4-oxopyrido[3,4-d]pyrimidin-3(4H)-yl)ethoxy)phenyl)thieno[3,2-b]pyridin-3-yl)acetate. (Example 1) as a yellow solid. Yield: 0.023 g, 10.0%; MS (ESI) m/z 828.41 [M+1]⁺. ¹H NMR (400 MHz, DMSO-d6) δ 8.72 (s, 1H), 8.70 (d, J=4.8 Hz, 1H), 7.72 (s, 1H), 7.54 (dd, J=8.8, 2.8 Hz, 1H), 7.41 (d, J=2.4 Hz, 1H), 7.32-7.19 (m, 6H), 4.36 (t, J=4.0 Hz, 2H), 4.22-4.18 (m, 5H), 3.06 (s, 3H), 2.93-2.87 (m, 2H), 2.74-2.64 (m, 3H), 2.40-2.32 (m, 2H), 1.93-1.71 (m, 9H).

### Example 2

### 2,4-difluorophenyl 2-(7-(5-chloro-2-(2-(5-cyano-6-((1-(3,3-difluorocyclobutyl) piperidin-4-yl)(methyl)amino)-2-methyl-4-oxopyrido[3,4-d]pyrimidin-3(4H)-yl) ethoxy)phenyl) thieno[3,2-b]pyridin-3-yl)acetate (Example 2)

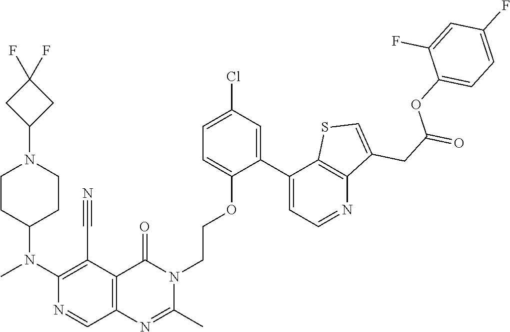 [SMILES: Cc1nc2cnc(N(C)C3CCN(C4CC(F)(F)C4)CC3)c(C#N)c2c(=O)n1CCOc1ccc(Cl)cc1-c1ccnc2c(CC(=O)Oc3ccc(F)cc3F)csc12]

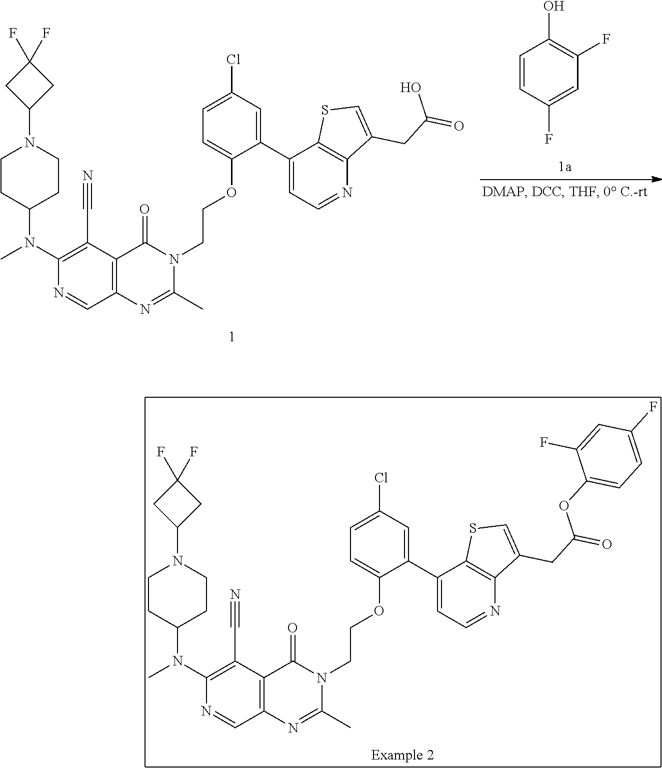 [SMILES: Cc1nc2cnc(N(C)C3CCN(C4CC(F)(F)C4)CC3)c(C#N)c2c(=O)n1CCOc1ccc(Cl)cc1-c1ccnc2c(CC(=O)O)csc12 ; Oc1ccc(F)cc1F ; Cc1nc2cnc(N(C)C3CCN(C4CC(F)(F)C4)CC3)c(C#N)c2c(=O)n1CCOc1ccc(Cl)cc1-c1ccnc2c(CC(=O)Oc3ccc(F)cc3F)csc12]

### Synthesis of 2,4-difluorophenyl 2-(7-(5-chloro-2-(2-(5-cyano-6-((1-(3,3-difluorocyclobutyl) piperidin-4-yl)(methyl)amino)-2-methyl-4-oxopyrido[3,4-d]pyrimidin-3(4H)-yl) ethoxy)phenyl) thieno[3,2-b]pyridin-3-yl)acetate. (Example 2)

A solution of 2-(7-(5-chloro-2-(2-(5-cyano-6-((1-(3,3-difluorocyclobutyl)piperidin-4-yl)(methyl)amino)-2-methyl-4-oxopyrido[3,4-d]pyrimidin-3(4H)-yl)ethoxy)phenyl)thieno[3,2-b]pyridin-3-yl)acetic acid (1, 0.20 g, 0.272 moll) in tetrahydrofuran (4 mL) was cooled to 0° C. 2,4-difluorophenol (la, 0.035 g, 0.272 moll), N,N′-dicyclohexylcarbodiimide (0.067 g, 0.327 mmol) and 4-dimethylaminopyridine (0.099 g, 0.818 mmol) were added and the mixture was stirred at room temperature for 3 h. After completion, the reaction mixture was diluted with ethyl acetate, washed with water and 5% aqueous citric acid solution. The organic layer was dried over anhydrous sodium sulphate, filtered and concentrated. The crude product was purified by preparative HPLC to afford 2,4-difluorophenyl 2-(7-(5-chloro-2-(2-(5-cyano-6-((1-(3,3-difluorocyclobutyl) piperidin-4-yl)(methyl)amino)-2-methyl-4-oxopyrido[3,4-d]pyrimidin-3(4H)-yl) ethoxy)phenyl) thieno[3,2-b]pyridin-3-yl)acetate. (Example 2) as an yellow solid. Yield: 0.013 g, 5.0%; MS (ESI) m/z 846.50 [M+1]⁺. ¹H NMR (400 MHz, DMSO-d6) δ 8.73 (s, 1H), 8.70 (d, J=4.4, 2H), 7.74 (s, 1H), 7.55 (dd, J=8.8, 2.8 Hz, 1H), 7.50-7.27 (m, 5H), 7.18-7.13 (m, 1H), 4.36 (s, 2H), 4.30 (s, 2H), 4.19 (s, 3H), 3.06 (s, 3H), 2.89 (s, 2H), 2.73-2.63 (m, 3H), 2.44-2.32 (m, 2H), 1.90-1.77 (m, 6H), 1.71 9s, 3H).

### Example 3

### 7-(5-chloro-2-(3-(5-cyano-6-((1-(3,3-difluorocyclobutyl)piperidin-4-yl)(methyl)amino)-2-methyl-4-oxopyrido[3,4-d]pyrimidin-3(4H)-yl)prop-1-yn-1-yl)phenyl)-N-((12-(N-methylacrylamido)dodecyl)sulfonyl)thieno[3,2-b]pyridine-3-carboxamide.trifluoroacetic acid (Example 3)

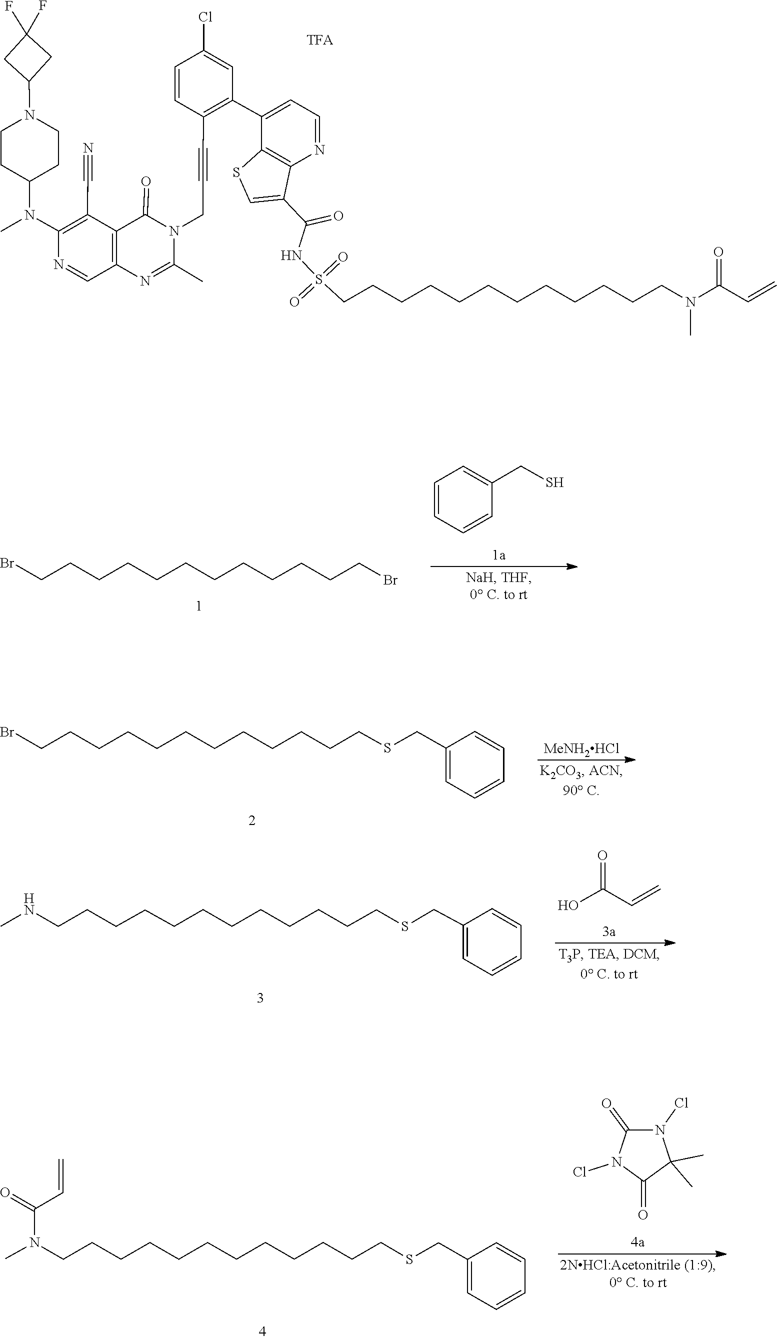

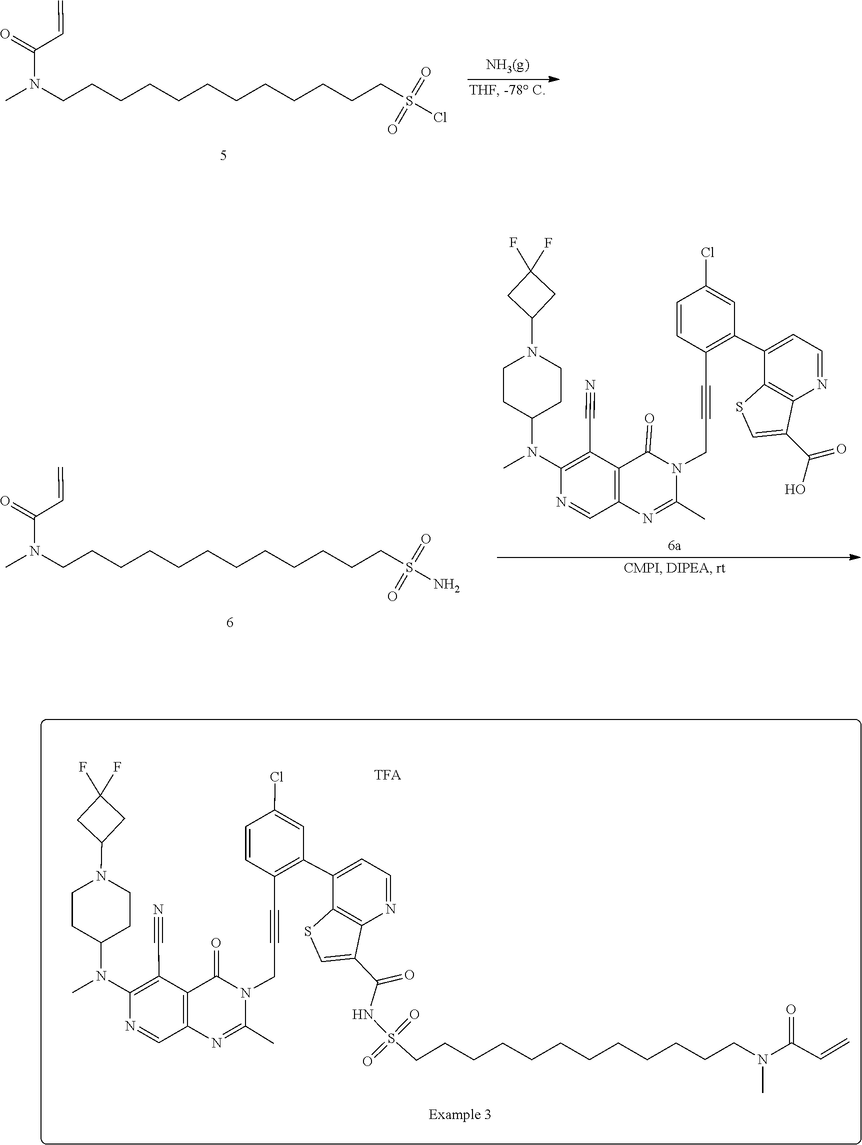 [SMILES: C=CC(=O)N(C)CCCCCCCCCCCCS(=O)(=O)Cl ; C=CC(=O)N(C)CCCCCCCCCCCCS(N)(=O)=O ; Cc1nc2cnc(N(C)C3CCN(C4CC(F)(F)C4)CC3)c(C#N)c2c(=O)n1CC#Cc1ccc(Cl)cc1-c1ccnc2c(C(=O)O)csc12 ; C=CC(=O)N(C)CCCCCCCCCCCCS(=O)(=O)NC(=O)c1csc2c(-c3cc(Cl)ccc3C#CCn3c(C)nc4cnc(N(C)C5CCN(C6CC(F)(F)C6)CC5)c(C#N)c4c3=O)ccnc12]

### Synthesis of benzyl(12-bromododecyl)sulfane (2)

To a solution of phenylmethanethiol (la, 1.62 g, 18.28 mmol) in tetrahydrofuran (100 mL), sodium hydride (60% in mineral oil, 1.21 g, 30.47) was added protion wise at 0° C. and the mixture was stirred for 20 minutes. Then, 1,12-dibromododecane (5.00 g, 15.23 mmol) was added drop wise and the mixture was stirred at room temperature for 2 h. After completion, the reaction mixture was quenched with cold water and extracted with ethyl acetate. The organic layer was washed with water and brine, dried over anhydrous sodium sulphate, filtered and concentrated. The crude product was purified by column chromatography (100-200 silica gel) using 0-3% ethyl acetate in n-hexane as an eluent. The desired fractions were concentrated to afford benzyl(12-bromododecyl)sulfane (2) as colourless liquid. Yield: 4.00 g, 27%, ¹H NMR (400 MHz, CDCl3 7.30-7.22 (m, 5H), 3.70 (s, 2H), 3.53-3.50 (m, 2H), 2.38-2.35 (m, 2H), 1.81-1.74 (m, 2H), 1.48-1.36 (m, 4H), 1.24-1.22 (m, 14H).

### Synthesis of 12-(benzylthio)-N-methyldodecan-1-amine (3)

To a solution of benzyl(12-bromododecyl)sulfane (2, 3.00 g, 8.07 mmol) in acetonitrile (30 mL). potassium carbonate (3.34 g, 24.23 mmol) and methanamine hydrochloride (1.62 g, 24.23 mmol) were added and the mixture was stirred at 90° for 16 h. After completion, the reaction mixture was diluted with water and extracted with ethyl acetate. The combined organic layer was washed with water and brine, dried over anhydrous sodium sulphate, filtered and concentrated. The crude product was purified by column chromatography (100-200 silica gel) using 0-10% methanol in dichloromethane as an eluent. The desired fractions were concentrated to afford 12-(benzylthio)-N-methyldodecan-1-amine (3) as an off white solid. Yield: 1.50 g, 59%, MS (ESI) m/z 322.39 [M+1]⁺.

### Synthesis of N-(12-(benzylthio)dodecyl)-N-methylacrylamide (4)

To a solution of 12-(benzylthio)-N-methyldodecan-1-amine (3, 1.50 g, 4.66 mmol) in dichloromethane (20.0 mL), acrylic acid (0.40 g, 5.59 mmol) was added and the reaction mixture was cooled to 0° C. Triethylamine (1.96 mL, 13.99 mmol) was added followed by propylphosphonic acid anhydride (50% solution in ethyl acetate, 8.90 mL, 13.99 mmol) drop wise and the mixture was stirred at 0° C. under nitrogen atmosphere for 1 h. After completion, the reaction mixture was diluted with dichloromethane, washed with water and brine, dried over anhydrous sodium sulphate, filtered and concentrated. The crude product was purified by column chromatography (100-200 silica gel) using 0-5% methanol in dichloromethane as an eluent. The desired fractions were concentrated to afford N-(12-(benzylthio)dodecyl)-N-methylacrylamide (4) as an off white solid. Yield: 0.60 g, 35%, MS (ESI) m/z 376.20 [M+1]⁺.

### Synthesis of 12-(N-methylacrylamido)dodecane-1-sulfonyl chloride (5)

A solution of N-(12-(benzylthio)dodecyl)-N-methylacrylamide (4, 0.60 g, 1.56 mmol) in acetonitrile (9 mL), and 2N hydrochloric acid (1.00 mL) was cooled to 0° C., 1,3-dichloro-5,5-dimethylimidazolidine-2,4-dione (0.62 g, 3.12 mmol) was added and the mixture was stirred at 0° C. for 1 h. After completion, the reaction mixture was quenched with saturated sodium bicarbonate solution and extracted with dichloromethane. The combined organic layer was washed with brine, dried over anhydrous sodium sulphate, filtered and concentrated to afford 12-(N-methylacrylamido)dodecane-1-sulfonyl chloride (5) as an off white low melting solid which was forwarded for further step without any purification. Yield: 0.50 g, 89%. MS (ESI) m/z 350.36 [M+1]⁺.

### Synthesis of N-methyl-N-(12-sulfamoyldodecyl)acrylamide (6)

To a stirred solution of 12-(N-methylacrylamido) dodecane-1-sulfonyl chloride (5, 0.50 g, 1.42 mmol) in tetrahydrofuran (10.0 mL) at −78° C., ammonia gas was purged for 15 minutes and the reaction mixture was allowed to warm to 0° C. over an hour. After completion, quenched with ice cold water and extracted with 5% methanol in dichloromethane solution. The combined organic layer was dried over anhydrous sodium sulphate, filtered and concentrated. The crude product was purified by column chromatography (100-200 silica gel) using 0-10% methanol in dichloromethane as an eluent. The desired fractions were concentrated to afford N-methyl-N-(12-sulfamoyldodecyl) acrylamide (6) as an off white solid. Yield: 0.15 g, 32%. MS (ESI) m/z 33.24 [M+1]⁺.

### Synthesis of 7-(5-chloro-2-(3-(5-cyano-6-((1-(3,3-difluorocyclobutyl)piperidin-4-yl)(methyl)amino)-2-methyl-4-oxopyrido[3,4-d]pyrimidin-3(4H)-yl)prop-1-yn-1-yl)phenyl)-N-((12-(N-methylacrylamido)dodecyl)sulfonyl)thieno[3,2-b]pyridine-3-carboxamide.trifluroracetic acid (Example 3)

To a solution of 7-(5-chloro-2-(3-(5-cyano-6-((1-(3,3-difluorocyclobutyl)piperidin-4-yl)(methyl)amino)-2-methyl-4-oxopyrido[3,4-d]pyrimidin-3(4H)-yl)prop-1-yn-1-yl)phenyl)thieno[3,2-b]pyridine-3-carboxylic acid trifluoroacetic acid (6a, 0.10 g, 0.14 mmol) in acetonitrile (3.0 mL), diisopropylethyl amine (0.12 mL, 0.70 mmol), and 2-chloro-1-methylpyridinium iodide (0.13 g, 0.42 mmol) were successively added at 0° C. and the mixture was stirred at room temperature for 1 h. Then, N-methyl-N-(12-sulfamoyldodecyl) acrylamide (6, 0.093 g, 0.28 mmol) was added and the mixture was stirred at room temperature for 16 h. After completion, the reaction mixture was diluted with dichloromethane, washed with water and 10% aqueous citric acid solution, dried over anhydrous sodium sulphate, filtered and concentrated. The crude product was purified by preparative HPLC to afford 7-(5-chloro-2-(3-(5-cyano-6-((1-(3,3-difluorocyclobutyl)piperidin-4-yl)(methyl)amino)-2-methyl-4-oxopyrido[3,4-d]pyrimidin-3(4H)-yl)prop-1-yn-1-yl)phenyl)-N-((12-(N-methylacrylamido)dodecyl)sulfonyl)thieno[3,2-b]pyridine-3-carboxamide.trifluoroacetic acid (Example 3) as a yellow solid. Yield: 0.03 g, 21%. MS (ESI) m/z 1028.63 [M+1]⁺. ¹H NMR (400 MHz, DMSO-d6) δ 12.33 (bs, 1H), 8.81 (s, 1H), 8.79 (d, J=4.80 Hz, 1H), 8.71 (s, 1H), 8.70 (s, 1H), 7.75 (d, J=8.4 Hz, 1H), 7.68-7.62 (m, 2H), 7.58 (d, J=4.4 Hz, 1H), 6.66 (dd, J=16.8, 10.4 Hz, 1H), 6.05 (dd, J=16.8, 2.4 Hz, 1H), 5.59 (dd, J=10.4, 2.4 Hz, 1H), 4.84 (s, 2H), 4.36-4.30 (m, 1H), 3.64 (t, J=7.6 Hz, 2H), 3.31 (t, J=7.2 Hz, 2H), 3.12 (s, 3H), 3.00-2.92 (m, 5H), 2.80-2.66 (m, 2H), 2.45-2.35 (m, 2H), 2.10-1.98 (m, 5H), 1.98-1.79 (m, 6H), 1.50-1.40 (m, 4H), 1.35-1.1.5 (m, 15H).

### Example 4

### 7-(5-chloro-2-(3-(5-cyano-6-((1-(3,3-difluorocyclobutyl)piperidin-4-yl)(methyl)amino)-2-methyl-4-oxopyrido[3,4-d]pyrimidin-3(4H)-yl)prop-1-yn-1-yl)phenyl)-N-((8-(N-methylacrylamido)octyl)sulfonyl)thieno[3,2-b]pyridine-3-carboxamide.trifluoroacetic acid (Example 4)

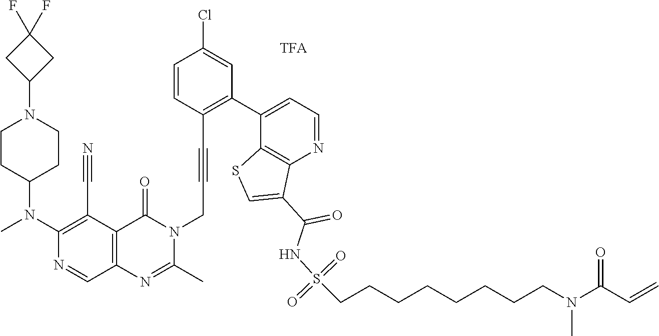 [SMILES: C=CC(=O)N(C)CCCCCCCCS(=O)(=O)NC(=O)c1csc2c(-c3cc(Cl)ccc3C#CCn3c(C)nc4cnc(N(C)C5CCN(C6CC(F)(F)C6)CC5)c(C#N)c4c3=O)ccnc12]

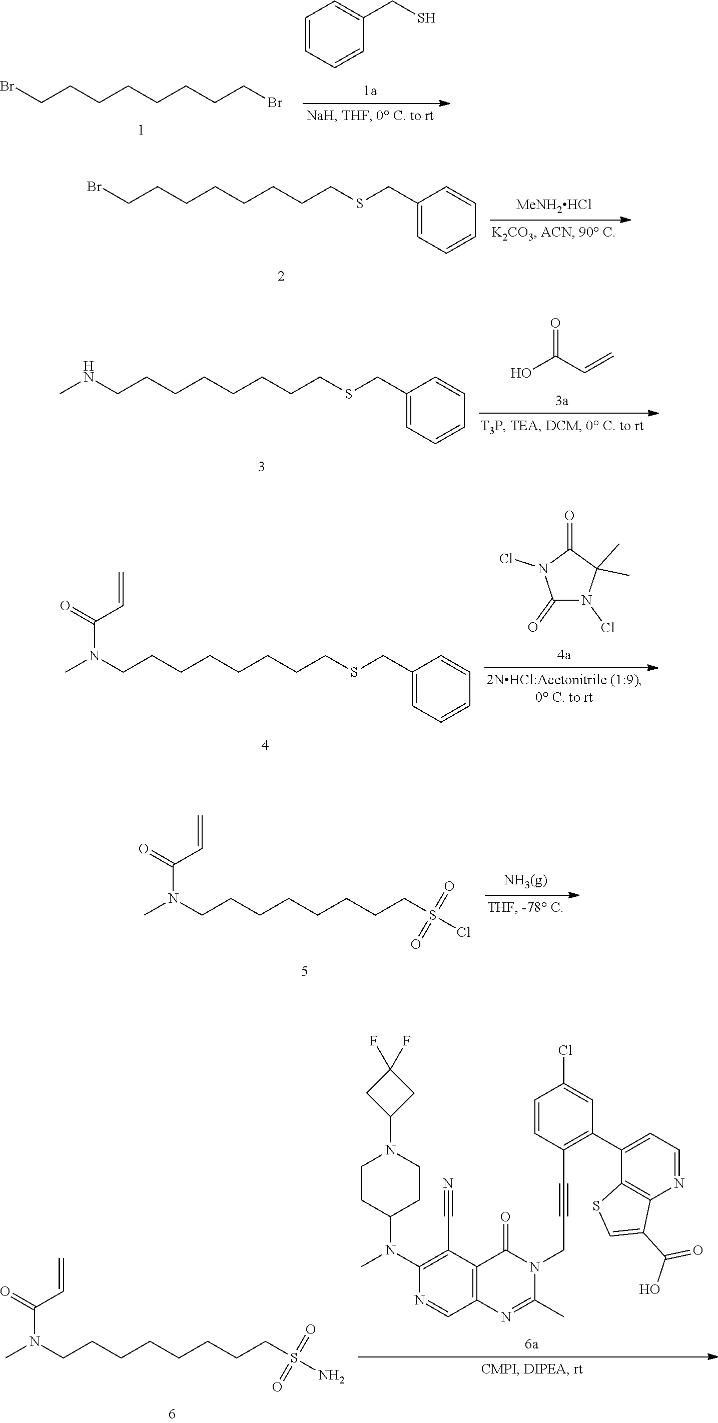

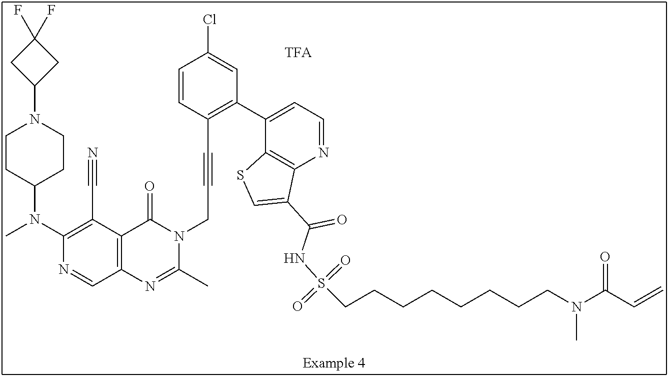 [SMILES: C=CC(=O)N(C)CCCCCCCCS(=O)(=O)NC(=O)c1csc2c(-c3cc(Cl)ccc3C#CCn3c(C)nc4cnc(N(C)C5CCN(C6CC(F)(F)C6)CC5)c(C#N)c4c3=O)ccnc12]

### Synthesis of benzyl(8-bromooctyl)sulfane (2)

To a solution of phenylmethanethiol (la, 1.62 g, 13.23 mmol) in tetrahydrofuran (50 mL), sodium hydride (60% in mineral oil, 0.88 g, 22.04) was added lot wise at 0° C. and the mixture was stirred for 20 minutes. Then 1,8-dibromooctane (3.00 g, 11.02 mmol) was added dropwise in to the reaction mixture and the mixture was stirred at room temperature for 2 h. After completion, the reaction mixture was quenched with cold water and extracted with ethyl acetate. The organic layer was washed with water and brine, dried over anhydrous sodium sulphate, filtered and concentrated. The crude product was purified by column chromatography (100-200 silica gel) using 0-5% ethyl acetate in n-hexane as an eluent. The desired fractions were concentrated to afford benzyl (8-bromooctyl)sulfane (2) as colourless liquid. Yield: 3.00 g, 86%, ¹HNMR (400 MHz, CDCl3 7.31-7.23 (m, 5H), 3.69 (s, 2H), 3.41-3.38 (m, 2H), 2.42-2.38 (m, 2H), 1.89-1.80 (m, 2H), 1.54-1.28 (m, 10H).

### Synthesis of 8-(benzylthio)-N-methyloctan-1-amine (3)

To a solution of benzyl(8-bromooctyl)sulfane (2, 3.00 g, 9.51 mmol) in acetonitrile (30 mL), potassium carbonate (3.94 g, 28.54 mmol) and methanamine hydrochloride (1.91 g, 28.54 mmol) were added and the mixture was heated at 90° C. for 16 h. After completion, the reaction mixture was diluted with water and extracted with ethyl acetate. The combined organic layer was washed with water and brine, dried over anhydrous sodium sulphate, filtered and concentrated. The crude product was purified by column chromatography (100-200 silica gel) using 0-10% methanol in dichloromethane as an eluent. The desired fractions were concentrated to afford 8-(benzylthio)-N-methyloctan-1-amine (3) as an off white solid. Yield: 2.10 g, 84%, MS (ESI) m/z 266.05 [M+1]⁺.

### Synthesis of N-(8-(benzylthio)octyl)-N-methylacrylamide (4)

To a solution of 8-(benzylthio)-N-methyloctan-1-amine (3, 2.00 g, 7.53 mmol) in dichloromethane (30.0 mL), acrylic acid (0.81 g, 11.30 mmol) was added and the reaction mixture was cooled at 0° C. Then, triethylamine (3.20 mL, 22.55 mmol) was added followed by propylphosphonic acid anhydride (50% solution in ethyl acetate, (14.39 mL, 22.64 mmol) dropwise into the reaction and the mixture was stirred at 0° C. under nitrogen atmosphere for 1 h. After completion, the reaction mixture was diluted with dichloromethane, washed with water, and brine, dried over anhydrous sodium sulphate, filtered and concentrated. The crude product was purified by column chromatography (100-200 silica gel) using 0-5% methanol in dichloromethane as an eluent. The desired fractions were concentrated to afford N-(8-(benzylthio)octyl)-N-methylacrylamide (4) as an off white solid. Yield: 1.00 g, 42%, MS (ESI) m/z 320.32 [M+1]⁺.

### Synthesis of 8-(N-methylacrylamido)octane-1-sulfonyl chloride (5)

To a solution of N-(8-(benzylthio)octyl)-N-methylacrylamide (4, 0.50 g, 1.56 mmol) in acetonitrile (9 mL) and 2N hydrochloric acid (1.00 mL) at 0° C., 1,3-dichloro-5,5-dimethylimidazolidine-2,4-dione (0.46 g, 2.34 mmol) was added and the mixture was stirred at 0° C. for 1 h. After completion, the reaction mixture was quenched with saturated sodium bicarbonate solution and extracted with dichloromethane. The combined organic layer was washed with brine, dried over anhydrous sodium sulphate, filtered and concentrated to afford crude 8-(N-methylacrylamido)octane-1-sulfonyl chloride (5) as an off white low melting solid which was forwarded for further step without any purification. Yield: 0.50 g, crude (50%). MS (ESI) m/z 296.02 [M+1]⁺,]⁺.

### Synthesis of N-methyl-N-(8-sulfamoyloctyl)acrylamide (6)

To a solution of 8-(N-methylacrylamido)octane-1-sulfonyl chloride (5, 0.50 g, 1.69 mmol) in tetrahydrofuran (10.0 mL) at −78° C., ammonia gas was purged for 15 minutes and the reaction mixture was allowed to warm to 0° C. over an hour. After completion, the mixture was quenched with ice cold water and extracted with 5% methanol in dichloromethane solution. The combined organic layer was dried over anhydrous sodium sulphate, filtered and concentrated. The crude product was purified by column chromatography (100-200 silica gel) using 0-5% methanol in dichloromethane as an eluent. The desired fractions were concentrated to afford N-methyl-N-(8-sulfamoyloctyl)acrylamide (6) as an off white solid. Yield: 0.20 g, 43%. MS (ESI) m/z 277.08 [M+1]⁺.

### Synthesis of 7-(5-chloro-2-(3-(5-cyano-6-((1-(3,3-difluorocyclobutyl)piperidin-4-yl)(methyl)amino)-2-methyl-4-oxopyrido[3,4-d]pyrimidin-3(4H)-yl)prop-1-yn-1-yl)phenyl)-N-((8-(N-methylacrylamido)octyl)sulfonyl)thieno[3,2-b]pyridine-3-carboxamide.trifluoroacetic acid (Example 4)

A solution of 7-(5-chloro-2-(3-(5-cyano-6-((1-(3,3-difluorocyclobutyl)piperidin-4-yl)(methyl)amino)-2-methyl-4-oxopyrido[3,4-d]pyrimidin-3(4H)-yl)prop-1-yn-1-yl)phenyl)thieno[3,2-b]pyridine-3-carboxylic acid trifluoroacetic acid (6a, 0.12 g, 0.17 mmol) in acetonitrile (3.0 mL) was cooled to 0° C., diisopropylethylamine (0.15 mL, 0.85 mmol) and 2-Chloro-1-methylpyridinium iodide (0.13 g, 0.51 mmol) were added and the mixture was stirred at room temperature for 1 h. Then, N-methyl-N-(8-sulfamoyloctyl) acrylamide (0.093 g, 0.33 mmol) was added and the mixture was stirred at room temperature for 16 h. After completion, the reaction mixture was diluted with dichloromethane, washed with water and 10% aqueous citric acid solution, dried over anhydrous sodium sulphate, filtered and concentrated. The crude product was purified by preparative HPLC to afford 7-(5-chloro-2-(3-(5-cyano-6-((1-(3,3-difluorocyclobutyl)piperidin-4-yl)(methyl)amino)-2-methyl-4-oxopyrido[3,4-d]pyrimidin-3(4H)-yl)prop-1-yn-1-yl)phenyl)-N-((8-(N-methylacrylamido)octyl)sulfonyl)thieno[3,2-b]pyridine-3-carboxamide.trifluoroacetic acid (Example 4) as a yellow solid. Yield: 0.01 g, 6%. MS (ESI) m/z 972.55 [M+1]⁺; ¹H NMR (400 MHz, DMSO-d6) δ 12.35 (s, 1H), 8.82-8.78 (m, 2H), 8.71 (s, 1H), 7.74 (d, J=8.4 Hz, 1H), 7.67-7.63 (m, 2H), 7.57 (d, J=4.4 Hz, 1H), 6.65 (dd, J=16.8, 10.4 Hz, 1H), 6.04 (dd, J=16.8, 2.4 Hz, 1H), 5.58 (dd, J=10.4, 2.4 Hz, 1H), 4.83 (s, 2H), 4.32-4.29 (m, 1H), 3.70-3.60 (m, 2H), 3.39 (t, J=7.2 Hz, 2H), 3.12 (s, 3H), 3.00-2.80 (m, 5H), 2.80-2.65 (m, 2H), 2.45-2.35 (m, 2H), 2.05-1.98 (m, 5H), 1.98-1.79 (m, 7H), 1.50-1.40 (m, 4H), 1.35-1.1.5 (m, 6H).

### Example 5

### 7-(5-chloro-2-(3-(5-cyano-6-((1-(3,3-difluorocyclobutyl)piperidin-4-yl)(methyl)amino)-2-methyl-4-oxopyrido[3,4-d]pyrimidin-3(4H)-yl)prop-1-yn-1-yl)phenyl)-N-(vinylsulfonyl)thieno[3,2-b]pyridine-3-carboxamide trifluroacetic acid (Example 5)

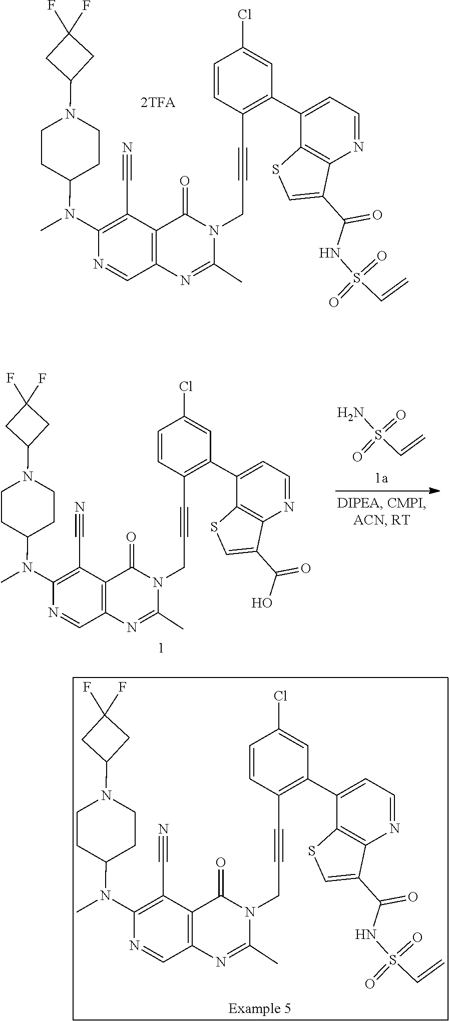

### Synthesis of 7-(5-chloro-2-(3-(5-cyano-6-((1-(3,3-difluorocyclobutyl)piperidin-4-yl)(methyl)amino)-2-methyl-4-oxopyrido[3,4-d]pyrimidin-3(4H)-yl)prop-1-yn-1-yl)phenyl)-N-(vinylsulfonyl)thieno[3,2-b]pyridine-3-carboxamide trifluroacetic acid (Example 5)

A solution of 7-(5-chloro-2-(3-(5-cyano-6-((1-(3,3-difluorocyclobutyl)piperidin-4-yl)(methyl)amino)-2-methyl-4-oxopyrido[3,4-d]pyrimidin-3(4H)-yl)prop-1-yn-1-yl)phenyl)thieno[3,2-b]pyridine-3-carboxylic acid (0.10 g, 0.140 mmol) in acetonitrile (3 mL) was added N,N-diisopropylethylamine (0.13 mL, 0.701 mmol) and 2-chloro-1-methylpyridinium iodide (0.071 g, 0.280 mmol) and the reaction mixture was stirred at room temperature for 30 minutes. Then, ethenesulfonamide (0.030 g, 0.280 mmol) was added and the reaction mixture was stirred at room temperature for 16 h. After completion, the reaction mixture was diluted with water and extracted with ethyl acetate. The organic layer was washed with brine and then dried over anhydrous sodium sulphate, filtered and concentrated. The crude was purified by preparative HPLC to afford 7-(5-chloro-2-(3-(5-cyano-6-((1-(3,3-difluorocyclobutyl)piperidin-4-yl)(methyl)amino)-2-methyl-4-oxopyrido[3,4-d]pyrimidin-3(4H)-yl)prop-1-yn-1-yl)phenyl)-N-(vinylsulfonyl)thieno[3,2-b]pyridine-3-carboxamide trifluroacetic acid (Example 5) as an yellow solid. Yield: 0.036 g, 32.14%; MS (ESI) m/z 803.37 [M+1]⁺; ¹H NMR (400 MHz, DMSO-d6) δ 12.60 (s, 1H), 9.88 (bs, 1H), 8.80 (d, J=4.8 Hz, 1H), 8.75 (s, 1H), 8.73 (s, 1H), 7.76 (d, J=8.00 Hz, 1H), 7.72-7.65 (m, 2H), 7.62 (d, J=4.8 Hz, 1H), 7.23 (d, J=16.8, 10.0 Hz, 1H), 6.50 (d, J=16.4 Hz, 1H), 6.38 (d, J=9.6 Hz, 1H), 4.82 (s, 2H), 4.60 (bs, 1H), 3.73 (s, 1H), 3.62-3.49 (m, 4H), 3.20-3.04 (m, 9H), 2.11 (bs, 4H), 1.97 (s, 3H).

### Example 6: Binding Studies of Synthesized Translational Inhibitor Compounds to eIF4E

One aspect of the invention is to characterize the binding of the 5 synthesized translational inhibitor compounds to eIF4E along with m7-GMP as positive control.

eIF4E is diluted 100 fold into 10 mM NaAc pH 5.0 with eT29 and m7-GMP each at 100 μM. This mixture is injected over a NHS/EDC activated CM7 chip and activated surfaces are blocked with 1 M ethanolamine resulting in the coupling of ˜25,000 RU of eIF4E. Similar coupling conditions are used to couple ˜27,000 RU of cytoplasmic carbonic anhydrase II (CAII) onto a second surface to serve as an off target.

m7-GMP is tested for binding using 20 μM as the highest concentration in a 2 fold dilution series. The m7-GMP binds only to the eIF4E surface, and not the CAII surface.

Next, eIF4E is coupled to a new CM5 sensor chip at two different densities. The stock NHS/EDC solutions are diluted 1:1 with water and injected for 5 mins. eIF4E is diluted 10 μL+10 eT29 (10 mM stock)+10 μL of m7-GMP (10 mM stock) with 1750 μL of 10 mM NaAc, pH 5.0. This solution is injected for 180 seconds and 90 seconds over the NHS/EDC activated surface to yield eIF4E coupling levels of ˜500 RU and 60 RU.

All five synthesized translational inhibitor compounds are assayed using a 3-fold dilution series from 40 nM up to 10 μM. The response data are processed using a reference spot without protein as well as a buffer injection. The processed data from the two different density eIF4E surfaces are globally fit to a 1:1 interaction model including a term for mass transport, and the binding constant of each translational inhibitor compound to eIF4E at 25° C. is determined.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the invention.

What is claimed is:

1. A translational inhibitor that binds covalently with eukaryotic initiation factor 4E (eIF4E), or a stereoisomer, tautomer or pharmaceutically acceptable salt of said translational inhibitor.

2. The translational inhibitor of claim 1, wherein the covalent binding of the translational inhibitor with eIF4E is reversible.

3. The translational inhibitor of claim 1, wherein the covalent binding of the translational inhibitor with eIF4E is irreversible.

4. The translational inhibitor of claim 1 that is an inhibitor of eIF4E.

5. The translational inhibitor of claim 1 that covalently binds with an amino acid residue at or near an active site of eIF4E.

6. The translational inhibitor of claim 5, wherein the amino acid residue at or near an active site of eIF4E is a charged amino acid residue or a polar amino acid residue.

7. The translational inhibitor of claim 6, wherein the amino acid residue at or near an active site of eIF4E is a cysteine residue.

8. The translational inhibitor of claim 6, wherein the amino acid residue at or near an active site of eIF4E is a lysine residue.

9. The translational inhibitor of claim 6, wherein the amino acid residue at or near an active site of eIF4E is an arginine residue.

10. The translational inhibitor of claim 6, wherein the amino acid residue at or near an active site of eIF4E is a serine residue.

11. The translational inhibitor of claim 1, wherein the inhibitor has a structure: 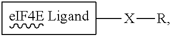
wherein X is a linker selected from a direct bond, alkylene, —O—, —S—, —NH—, —NH(alkylene)-, —NHSO₂—, —NHSO₂(alkylene)-, —C(O)NH—, —C(O)NH(alkylene)-, —C(O)NHSO₂—and —C(O)NHSO₂(alkylene)-, wherein alkyl and alkylene are unsubstituted or substituted; and
R is an electrophilic moiety.

12. The translational inhibitor of claim 11, wherein R is alkenyl, aryl, —C(O)₂alkyl,
—C(O)₂aryl, —NH(alkenyl), —N(alkyl)(alkenyl), —NHC(O)(alkenyl) or
—N(alkyl)C(O)(alkenyl), wherein alkyl, alkenyl and aryl are unsubstituted or substituted.

13. The translational inhibitor of claim 11, wherein the eIF4E Ligand has a structure according to Formula I, II, III, IV, V or VI.

14. The translational inhibitor of claim 11, wherein the translational inhibitor is

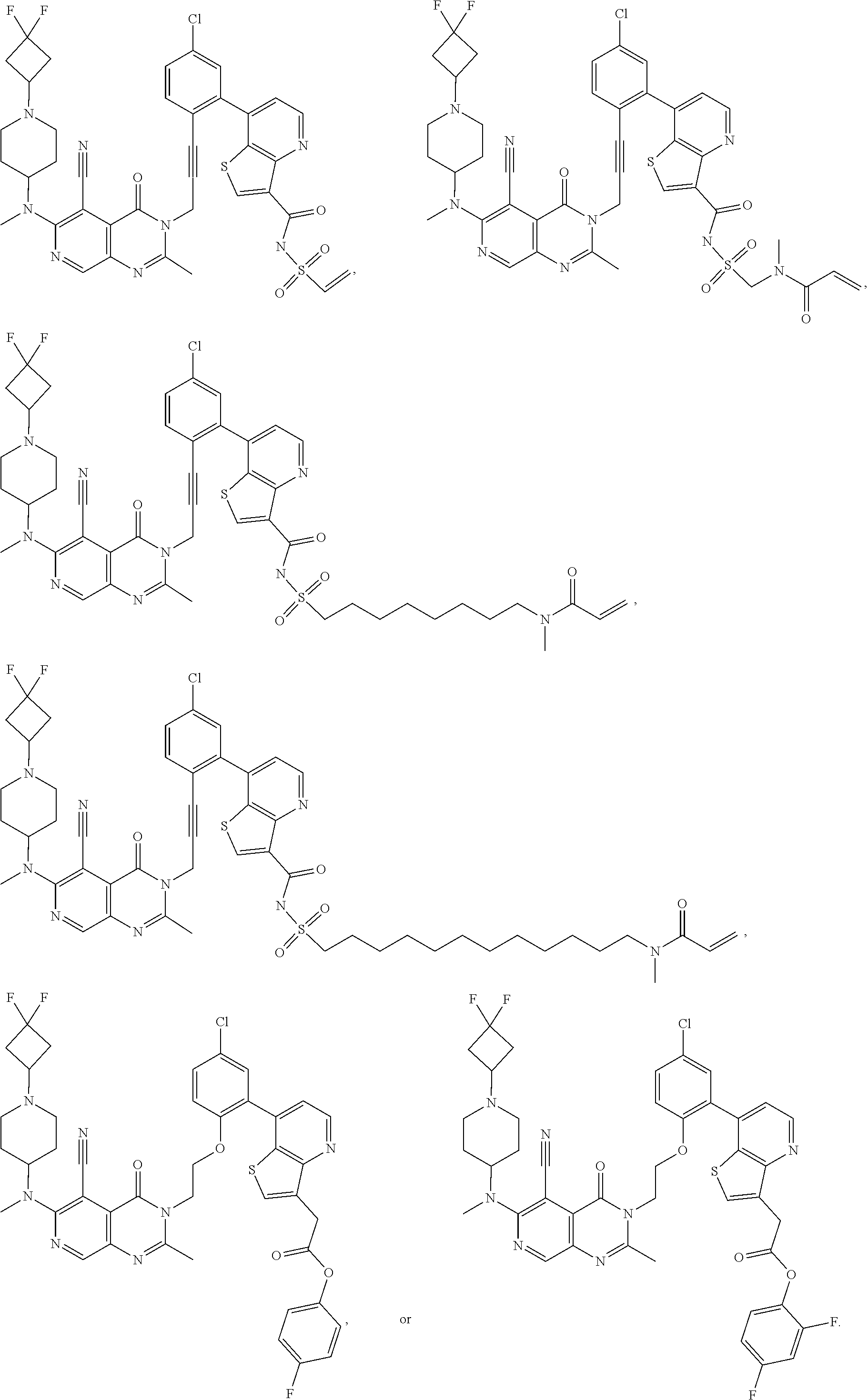

15. A pharmaceutical composition comprising (i) a therapeutically effective amount of the inhibitor of claim 1, or a stereoisomer, a tautomer or a pharmaceutically acceptable salt thereof, and (ii) one or more pharmaceutically acceptable carriers, diluents or excipients.

16. A method for treating a eIF4E-dependent condition in a subject in need thereof comprising administering to the subject (i) a therapeutically effective amount of the translational inhibitor of claim 1, or a stereoisomer, tautomer or pharmaceutically acceptable salt thereof, or (ii) a pharmaceutical composition of claim 11.

17. The method of claim 16, wherein the eIF4E-dependent condition is a condition selected from the group consisting of solid tumor, melanoma, multiple melanoma, non-small cell lung cancer, renal cell carcinoma, renal cancer, a hematological cancer, prostate cancer, castration-resistant prostate cancer, colon cancer, rectal cancer, gastric cancer, esophageal cancer, bladder cancer, head and neck cancer, thyroid cancer, breast cancer, triple-negative breast cancer, ovarian cancer, cervical cancer, lung cancer, urothelial cancer, pancreatic cancer, glioblastoma, hepatocellular cancer, myeloma, multiple myeloma, leukemia, B-cell lymphoma, T-cell lymphoma, hairy cell lymphoma, Hodgkin's lymphoma, non-Hodgkin's lymphoma, Burkitt's lymphoma, myelodysplastic syndrome, brain cancer, CNS cancer, malignant glioma, Alzheimer's, Parkinson's, Fragile X Syndrome, autism disorders, and any combination thereof.

18. The method of claim 16, wherein the translational inhibitor or pharmaceutical composition is administered via a route selected from the group consisting of oral, topical, transdermal, inhalation, parenteral, sublingual, buccal, rectal, vaginal, and intranasal routes.

19. A method for attenuating or inhibiting the activity of eIF4E in at least one cell overexpressing eIF4E, comprising contacting the at least one cell with the translational inhibitor of claim 1, or a stereoisomer, tautomer or pharmaceutically acceptable salt thereof.

20. The method of claim 19, wherein the at least one cell is a colon cancer cell, a gastric cancer cell, a thyroid cancer cell, a lung cancer cell, a leukemia cell, a B-cell lymphoma, a T-cell lymphoma, a hairy cell lymphoma, Hodgkin's lymphoma cell, non-Hodgkin's lymphoma cell, Burkitt's lymphoma cell, a pancreatic cancer cell, a melanoma cell, a multiple melanoma cell, a brain cancer cell, a CNS cancer cell, a renal cancer cell, a prostate cancer cell, an ovarian cancer cell, or a breast cancer cell.

21. A method for inhibiting translation in at least one cell overexpressing eIF4E, comprising contacting the at least one cell with the translational inhibitor of claim 1, or a stereoisomer, tautomer or pharmaceutically acceptable salt thereof.
